(19) United States
(12) Patent Application Publication
(10) Pub. No.: US 2023/0038355 A1
(43) Pub. Date: Feb. 9, 2023

(54) Manufacturing Methods for Producing Anti-IL12/IL23 Antibody Compositions

(71) Applicant: Janssen Biotech, Inc., Horsham, PA (US)

(72) Inventors: Ann Donlon, County Cork (IE); Charles Goochee, Ambler, PA (US); Ronan Hayes, County Cork (IE); Robin Johnston, County Cork (IE); Willem Steenvoorden, Zuid Holland (NL); Denis Twomey, County Cork (IE); Gary Gordon, County Cork (IE)

(21) Appl. No.: 17/860,453

(22) Filed: Jul. 8, 2022

Related U.S. Application Data
(60) Provisional application No. 63/219,904, filed on Jul. 9, 2021.

(51) Int. Cl. C07K 16/24; C12P 21/00; C12N 5/09

(52) U.S. Cl. CPC C07K 16/244; C12P 21/005; C12N 5/0694; C12N 2500/32; C12N 2500/02; C12N 2500/40

(57) ABSTRACT

The present disclosure relates to methods of manufacture for producing anti-IL-12/IL-23p40 antibodies, e.g., the anti-IL-12/IL-23p40 antibody ustekinumab, and specific pharmaceutical compositions of the antibody.

### CROSS-REFERENCE TO RELATED APPLICATIONS

This application claims priority to U.S. Provisional Application Ser. No. 63/219,904, filed 9 Jul. 2021. The entire contents of the aforementioned application are incorporated herein by reference in their entireties.

### REFERENCE TO SEQUENCE LISTING SUBMITTED ELECTRONICALLY

This application contains a sequence listing, which is submitted electronically via The United States Patent and Trademark Center Patent Center as an XML formatted sequence listing with a file name “JBI6005 Sequence Listing.xml” and a creation date of Jun. 29, 2022, and having a size of 12 Kb. The sequence listing submitted via Patent Center is part of the specification and is herein incorporated by reference in its entirety

The present invention relates to methods of manufacture for producing anti-IL-12/IL-23p40 antibodies, e.g., the anti-IL-12/IL-23p40 antibody ustekinumab, and specific pharmaceutical compositions of the antibody.

### BACKGROUND OF THE INVENTION

### Field of the Invention

Interleukin (IL)-12 is a secreted heterodimeric cytokine comprised of 2 disulfide-linked glycosylated protein subunits, designated p35 and p40 for their approximate molecular weights. IL-12 is produced primarily by antigen-presenting cells and drives cell-mediated immunity by binding to a two-chain receptor complex that is expressed on the surface of T cells or natural killer (NK) cells. The IL-12 receptor beta-1 (IL-12Rβ1) chain binds to the p40 subunit of IL-12, providing the primary interaction between IL-12 and its receptor. However, it is IL-12p35 ligation of the second receptor chain, IL-12Rβ2, that confers intracellular signaling (e.g. STAT4 phosphorylation) and activation of the receptor-bearing cell (Presky et al, 1996). IL-12 signaling concurrent with antigen presentation is thought to invoke T cell differentiation towards the T helper 1 (Th1) phenotype, characterized by interferon gamma (IFNγ) production (Trinchieri, 2003). Th1 cells are believed to promote immunity to some intracellular pathogens, generate complement-fixing antibody isotypes, and contribute to tumor immunosurveillance. Thus, IL-12 is thought to be a significant component to host defense immune mechanisms.

It was discovered that the p40 protein subunit of IL-12 can also associate with a separate protein subunit, designated p19, to form a novel cytokine, IL-23 (Oppman et al, 2000). IL-23 also signals through a two-chain receptor complex. Since the p40 subunit is shared between IL-12 and IL-23, it follows that the IL-12Rβ1 chain is also shared between IL-12 and IL-23. However, it is the IL-23p19 ligation of the second component of the IL-23 receptor complex, IL-23R, that confers IL-23 specific intracellular signaling (e.g., STAT3 phosphorylation) and subsequent IL-17 production by T cells (Parham et al, 2002; Aggarwal et al. 2003). Recent studies have demonstrated that the biological functions of IL-23 are distinct from those of IL-12, despite the structural similarity between the two cytokines (Langrish et al, 2005).

Abnormal regulation of IL-12 and Th1 cell populations has been associated with many immune-mediated diseases since neutralization of IL-12 by antibodies is effective in treating animal models of psoriasis, multiple sclerosis (MS), rheumatoid arthritis, inflammatory bowel disease, insulin-dependent (type 1) diabetes mellitus, and uveitis (Leonard et al, 1995; Hong et al, 1999; Malfait et al, 1998; Davidson et al, 1998). IL-12 has also been shown to play a critical role in the pathogenesis of SLE in two independent mouse models of systemic lupus erythematosus (Kikawada et al. 2003; Dai et al. 2007.

Systemic lupus erythematosus (SLE) is a complex, chronic, heterogeneous autoimmune disease of unknown etiology that can affect almost any organ system, and which follows a waxing and waning disease course. Systemic lupus erythematosus occurs much more often in women than in men, up to 9 times more frequently in some studies, and often appears during the child-bearing years between ages 15 and 45. This disease is more prevalent in Afro-Caribbean, Asian, and Hispanic populations. In SLE, the immune system attacks the body's cells and tissue, resulting in inflammation and tissue damage which can harm the heart, joints, skin, lungs, blood vessels, liver, kidneys and nervous system. About half of the subjects diagnosed with SLE present with organ-threatening disease, but it can take several years to diagnose subjects who do not present with organ involvement. Some of the primary complaints of newly diagnosed lupus patients are arthralgia (62%) and cutaneous symptoms (new photosensitivity; 20%), followed by persistent fever and malaise.³⁹ The estimated annual incidence of lupus varies from 1.8 to 7.6 cases per 100,000 and the worldwide prevalence ranges from 14 to 172 cases per 100,000 people.³⁹ Patients with mild disease have mostly skin rashes and joint pain and require less aggressive therapy; regimens include nonsteroidal anti-inflammatory drugs (NSAIDs), anti-malarials (e.g., hydroxychloroquine, chloroquine, or quinacrine) and/or low dose corticosteroids. With more severe disease patients may experience a variety of serious conditions depending on the organ systems involved, including lupus nephritis with potential renal failure, endocarditis or myocarditis, pneumonitis, pregnancy complications, stroke, neurological complications, vasculitis and cytopenias with associated risks of bleeding or infection. Common treatments for more severe disease include immunomodulatory agents, such as methotrexate (MTX), azathioprine, cyclophosphamide, cyclosporine, high dose corticosteroids, biologic B cell cytotoxic agents or B cell modulators, and other immunomodulators. Patients with serious SLE have a shortening of life expectancy by 10 to 30 years, largely due to the complications of the disease, of standard of care therapy, and/or accelerated atherosclerosis. In addition, SLE has a substantial impact on quality of life, work productivity, and healthcare expenditures. Existing therapies for SLE are generally either cytotoxic or immunomodulatory, and may have notable safety risks. Newer treatments for SLE have provided only modest benefits over standard of care therapy. Thus, there is a large unmet need for new alternative treatments that can provide significant benefit in this disease without incurring a high safety risk.

### SUMMARY OF THE INVENTION

The general and preferred embodiments are defined, respectively, by the independent and dependent claims appended hereto, which for the sake of brevity are incorporated by reference herein. Other preferred embodiments, features, and advantages of the various aspects of the invention will become apparent from the detailed description below taken in conjunction with the appended drawing figures.

In certain embodiments, the present invention provides anti-IL-12/23p40 antibodies comprising: (i) the heavy chain CDR amino acid sequences of SEQ ID NO:1, SEQ ID NO:2, and SEQ ID NO:3; and (ii) the light chain CDR amino acid sequences of SEQ ID NO:4, SEQ ID NO:5, and SEQ ID NO:6, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides anti-IL-12/23p40 antibodies comprising: (i) the heavy chain CDR amino acid sequences of SEQ ID NO:1, SEQ ID NO:2, and SEQ ID NO:3; and (ii) the light chain CDR amino acid sequences of SEQ ID NO:4, SEQ ID NO:5, and SEQ ID NO:6, wherein the anti-IL-12/23p40 antibodies are a follow-on biologic and wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides anti-IL-12/23p40 antibodies comprising: (i) the heavy chain variable domain amino acid sequence of SEQ ID NO:7; and (ii) the light chain variable domain amino acid sequence of SEQ ID NO:8, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides anti-IL-12/23p40 antibodies comprising: (i) the heavy chain amino acid sequence of SEQ ID NO:10; and (ii) the light chain amino acid sequence of SEQ ID NO:11, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides a method of manufacture for producing a drug substance (DS) or drug product (DP) comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies is controlled and the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, the method comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells.

In certain embodiments, the present invention provides a method of manufacture for producing a drug substance (DS) or drug product (DP) comprising anti-IL-12/23p40 antibodies comprising a heavy chain of the amino acid sequence of SEQ ID NO:10 and a light chain of the amino acid sequence of SEQ ID NO:11, wherein the anti-IL-12/23p40 antibodies are a follow-on biologic and wherein the oligosaccharide profile of the anti-IL-12/23p40 antibodies is controlled and the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, the method comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells.

In certain embodiments, the present invention provides a method of manufacture for producing a drug substance (DS) or drug product (DP) comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies is controlled and the peak 3 area % of the capillary isoelectric focusing (cIEF) electropherogram of the anti-IL-12/23p40 antibodies is ≥39.8% to ≤64.4%, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides a method of manufacture for producing a drug substance (DS) or drug product (DP) comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies is controlled and the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the specified trace metal concentrations of manganese and copper in the chemically defined medium are determined using inductively coupled plasma mass spectrometry (ICP-MS).

In certain embodiments, the present invention provides a method of manufacture for producing a drug substance (DS) or drug product (DP) comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies is controlled and the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, the method comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the trace metal concentrations of manganese and copper in the chemically defined medium are controlled by supplementing the chemically defined medium with one or more sources of manganese and copper, wherein the one or more sources of manganese are selected from the group consisting of: MnCl₂, MnSO₄, MnF₂ and MnI₂ and the one or more sources of copper are selected from the group consisting of: CuSO₄, CuCl₂, and Cu(OAc)₂.

In certain embodiments, the present invention provides a method of manufacture for producing a drug substance (DS) or drug product (DP) comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies is controlled and the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the oligosaccharide species are determined by high pressure liquid chromatography (HPLC).

In certain embodiments, the present invention provides a method of manufacture for producing a drug substance (DS) or drug product (DP) comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies is controlled and the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, the method comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the eukaryotic cells are selected from the group consisting of: Chinese hamster vary cells (CHO cells), human retinal cells (PER.C6 cells), and mouse myeloma cells (NS0 cells and Sp2/0 cells).

In certain embodiments, the present invention provides a composition comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies comprises total neutral oligosaccharide species ≥64.8% to ≤⁸5.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides a composition comprising anti-IL-12/23p40 antibodies comprising a heavy chain of the amino acid sequence of SEQ ID NO:10 and a light chain of the amino acid sequence of SEQ ID NO:11, wherein the anti-IL-12/23p40 antibodies are a follow-on biologic and wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides a composition comprising anti-IL-12/23p40 antibodies comprising a heavy chain of the amino acid sequence of SEQ ID NO:10 and a light chain of the amino acid sequence of SEQ ID NO:11, wherein the peak 3 area % of the capillary isoelectric focusing (cIEF) electropherogram of the anti-IL-12/23p40 antibodies is ≥39.8% to ≤64.4%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies.

In certain embodiments, the present invention provides a composition comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the specified trace metal concentrations of manganese and copper in the chemically defined medium are determined using inductively coupled plasma mass spectrometry (ICP-MS).

In certain embodiments, the present invention provides a composition comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the specified trace metal concentrations of manganese and copper in the chemically defined medium are controlled by supplementing the chemically defined medium with one or more sources of manganese and copper, wherein the one or more sources of manganese are selected from the group consisting of: MnCl₂, MnSO₄, MnF₂ and MnI₂ and the one or more sources of copper are selected from the group consisting of: CuSO₄, CuCl₂, and Cu(OAc)₂.

In certain embodiments, the present invention provides a composition comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the oligosaccharide species are determined by high pressure liquid chromatography (HPLC).

In certain embodiments, the present invention provides a composition comprising anti-IL-12/23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%, and wherein the anti-IL-12/23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/23p40 antibodies in the eukaryotic cells, wherein the concentrations of manganese and copper are effective at controlling the oligosaccharide profile of the anti-IL-12/23p40 antibodies, and wherein the eukaryotic cells are selected from the group consisting of: Chinese hamster vary cells (CHO cells), human retinal cells (PER.C6 cells), and mouse myeloma cells (NS0 cells and Sp2/0 cells).

### BRIEF DESCRIPTION OF THE DRAWINGS

FIG. 1 shows an overview of the 10 stages of the ustekinumab manufacturing process.

FIG. 2 shows a flow diagram of Stage 1 manufacturing process for the preculture and expansion steps, including the in-process controls and process monitoring tests.

FIG. 3 shows a flow diagram of Stage 2 manufacturing process steps, including the in-process controls and process monitoring tests.

FIG. 4 shows a representative HPLC chromatogram for Oligosaccharide Analysis of ustekinumab.

FIG. 5 shows a representative cIEF electropherogram profile of ustekinumab. A graphic representing the general relationship between cIEF peaks and decreasing negative charge/degree of sialylation is also shown.

FIG. 6 shows a diagrammatic overview of some of the primary N-linked oligosaccharide species in ustekinumab IgG. The role of some of the enzymes in the glycosylation maturation process and role of some divalent cations (e.g. Mn²⁺ as a co-factor and Cu²⁺ as an inhibitor of GalTI) are also shown (see, e.g., Biotechnol Bioeng. 2007 Feb. 15; 96(3):538-49; Curr Drug Targets. 2008 April; 9(4):292-309; J Biochem Mol Biol. 2002 May 31; 35(3):330-6). Note that species with terminal sialic acid (S1 and S2) are charged species and species lacking the terminal sialic acid (G0F, G1F, and G2F) are neutral species, but generation of charged species depends on the presence of the galactose in G1F and G2F added by the GalTI enzyme.

FIG. 7 shows peak 3 area % for ustekinumab cIEF profile for pre-change, post-change, and SUP-AGT3 batches of ustekinumab displayed in chronological order for different batches. The mean % total for the historical pre-change AGT batches is shown with a solid line and the upper and lower specification limits are shown as dashed lines.

FIGS. 8A and 8B show total neutral (FIG. 8A) and total charged (FIG. 8B) oligosaccharide species in pre-change, post-change, and SUP-AGT3 batches of ustekinumab displayed in chronological order for different batches. The mean % totals for the historical pre-change AGT batches are shown with solid lines and the upper and lower specification limits are shown as dashed lines.

FIGS. 9A-9C show % of total for individual neutral oligosaccharide species G0F (FIG. 9A), G1F (FIG. 9B), and G2F (FIG. 9C) in pre-change, post-change, and SUP-AGT3 batches of ustekinumab displayed in chronological order for different batches. The mean % values for the historical pre-change AGT batches are shown with solid lines and the upper and lower specification limits are shown as dashed lines.

FIG. 10: shows manganese levels for different batches of ustekinumab in chronological order. The data are identified based on the media used, e.g., Pre-change AGT, Post-change AGT, and SUP-AGT3. The mean value for the historical batches is shown with a solid line and the upper and lower specification limits are shown as dashed lines.

FIG. 11: shows chromium levels for different batches of ustekinumab in chronological order. The data are identified based on the media used, e.g., Pre-change AGT, Post-change AGT, and SUP-AGT3.

FIG. 12: shows copper levels for different batches of ustekinumab in chronological order. The data are identified based on the media used, e.g., Pre-change AGT, Post-change AGT, and SUP-AGT3. The mean value for the historical batches is shown with a solid line and the upper and lower specification limits are shown as dashed lines. Note that many of the Post-change AGT batches had values less than the lower limit of the assay for copper (<1 μg/L) and are simply represented graphically as 1 μg/L.

FIG. 13: shows cell culture (Stage 2) viability (%) for cells in different 500-liter SUP-AGT3 batches of ustekinumab (solid circles, solid squares, empty squares, solid triangles, empty triangles, solid diamonds, and empty diamonds). The mean for historical Pre-change AGT batches is shown as a solid line and ±3SD for historical Pre-change AGT batches are shown as dashed lines.

FIG. 14: shows cell culture (Stage 2) cumulative IgG amounts (grams harvested) for different 500-liter SUP-AGT3 batches of ustekinumab (solid circles, solid squares, empty squares, solid triangles, empty triangles, solid diamonds, and empty diamonds). The mean for historical Pre-change AGT batches is shown as a solid line and ±3SD for historical Pre-change AGT batches are shown as dashed lines.

### DETAILED DESCRIPTION OF THE PREFERRED EMBODIMENTS

As used herein, an “anti-IL-12 antibody,” “anti-IL-23 antibody,” “anti-IL-12/23p40 antibody,” “anti-IL-12/IL-23p40 antibody,” “IL-12/23p40 antibody,” “IL-12/IL-23p40 antibody,” “antibody portion,” or “antibody fragment” and/or “antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-12 and/or IL-23 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-12/23 activity or binding, or with IL-12/23 receptor activity or binding, in vitro, in situ and/or in vivo. As a non-limiting example, a suitable anti-IL-12/23p40 antibody, specified portion or variant of the present invention can bind at least one IL-12/23 molecule, or specified portions, variants or domains thereof. A suitable anti-IL-12/23p40 antibody, specified portion, or variant can also optionally affect at least one of IL-12/23 activity or function, such as but not limited to, RNA, DNA or protein synthesis, IL-12/23 release, IL-12/23 receptor signaling, membrane IL-12/23 cleavage, IL-12/23 activity, IL-12/23 production and/or synthesis.

As used herein, the terms “antibody” or “antibodies”, include biosimilar antibody molecules approved under the Biologics Price Competition and Innovation Act of 2009 (BPCI Act) and similar laws and regulations globally. Under the BPCI Act, an antibody may be demonstrated to be biosimilar if data show that it is “highly similar” to the reference product notwithstanding minor differences in clinically inactive components and are “expected” to produce the same clinical result as the reference product in terms of safety, purity and potency (Endocrine Practice: February 2018, Vol. 24, No. 2, pp. 195-204). These biosimilar antibody molecules are provided an abbreviated approval pathway, whereby the applicant relies upon the innovator reference product's clinical data to secure regulatory approval. Compared to the original innovator reference antibody that was FDA approved based on successful clinical trials, a biosimilar antibody molecule is referred to herein as a “follow-on biologic.” As presented herein, STELARA® (ustekinumab) is the original innovator reference anti-IL-12/23p40 antibody that was FDA approved based on successful clinical trials. Ustekinumab has been on sale in the United States since 2009.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-12/23. For example, antibody fragments capable of binding to IL-12/23 or portions thereof, including, but not limited to, Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the C_{H}1 domain and/or hinge region of the heavy chain. The various portions of antibodies can be joined together chemically by conventional techniques, or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” may also be an antibody that is derived from or closely matches human germline immunoglobulin sequences. Human antibodies may include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Anti-IL-12/23p40 antibodies (also termed IL-12/23p40 antibodies) (or antibodies to IL-23) useful in the methods and compositions of the present invention can optionally be characterized by high affinity binding to IL-12/23p40 (or to IL-23) and, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-12 antibody in patients treated with anti-IL-12 antibody as occurring in less than 25% of patients treated, preferably, in less than 10% of patients treated with the recommended dose for the recommended course of therapy during the treatment period.

As used herein, the term “human antibody” refers to an antibody in which substantially every part of the protein (e.g., CDR, framework, C_{L}, C_{H} domains (e.g., C_{H}1, C_{H}2, and C_{H}3), hinge, (V_{L}, V_{H})) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally and preferably retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific antibodies e.g., DuoBody® (bispecific antibodies), heterospecific, heteroconjugate or similar antibodies can also be used that are monoclonal, preferably human or humanized, antibodies that have binding specificities for at least two different antigens. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, can be cumbersome with low product yields and different strategies have been developed to facilitate bispecific antibody production.

Full length bispecific antibodies can be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent monospecific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each can bind a distinct epitope.

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Intl. Publ. No. WO 2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by following substitutions (expressed as modified position in the first CH3 domain of the first heavy chain/modified position in the second CH3 domain of the second heavy chain): L351Y_F405A_Y407V/T394W, T3661K392M_T394W/F405A_Y407V, T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Pat. Publ. No. US2012/0149876 or U.S. Pat. Publ. No. US2013/0195849.

In addition to methods described above, bispecific antibodies can be generated in vitro in a cell-free environment by introducing asymmetrical mutations in the CH3 regions of two monospecific homodimeric antibodies and forming the bispecific heterodimeric antibody from two parent monospecific homodimeric antibodies in reducing conditions to allow disulfide bond isomerization according to methods described in Intl. Pat. Publ. No. WO2011/131746. In the methods, the first monospecific bivalent antibody and the second monospecific bivalent antibody are engineered to have certain substitutions at the CH3 domain that promoter heterodimer stability; the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol, preferably a reducing agent selected from the group consisting of: 2-mercaptoethylamine, dithiothreitol and tris(2-carboxyethyl)phosphine. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

The terms “efficacy” and “effective” as used herein in the context of a dose, dosage regimen, treatment or method refer to the effectiveness of a particular dose, dosage or treatment regimen. Efficacy can be measured based on change in the course of the disease in response to an agent of the present invention. For example, an anti-IL12/23p40 or anti-IL23 antibody of the present invention (e.g., the anti-IL12/23p40 antibody usetkinumab) is administered to a patient in an amount and for a time sufficient to induce an improvement, preferably a sustained improvement, in at least one indicator that reflects the severity of the disorder that is being treated. Various indicators that reflect the extent of the subject's illness, disease or condition may be assessed for determining whether the amount and time of the treatment is sufficient. Such indicators include, for example, clinically recognized indicators of disease severity, symptoms, or manifestations of the disorder in question. The degree of improvement generally is determined by a physician, who may make this determination based on signs, symptoms, biopsies, or other test results, and who may also employ questionnaires that are administered to the subject, such as quality-of-life questionnaires developed for a given disease.

The term “safe”, as it relates to a dose, dosage regimen, treatment or method with an anti-IL12/23p40 or anti-IL23 antibody of the present invention (e.g., the anti-IL12/23p40 antibody ustekinumab), refers to a favorable risk:benefit ratio with an acceptable frequency and/or acceptable severity of treatment-emergent adverse events (referred to as AEs or TEAEs) compared to the standard of care or to another comparator. An adverse event is an untoward medical occurrence in a patient administered a medicinal product. In particular, safe as it relates to a dose, dosage regimen or treatment with an anti-IL12/23p40 or anti-IL23 antibody of the present invention refers to with an acceptable frequency and/or acceptable severity of adverse events associated with administration of the antibody if attribution is considered to be possible, probable, or very likely due to the use of the anti-IL12/23p40 or anti-IL23 antibody.

### Utility

The isolated nucleic acids of the present invention can be used for production of at least one anti-IL-12/23p40 (or anti-IL-23) antibody or specified variant thereof, which can be used to measure or effect in an cell, tissue, organ or animal (including mammals and humans), to diagnose, monitor, modulate, treat, alleviate, help prevent the incidence of, or reduce the symptoms of, at least one IL-12/23 condition, selected from, but not limited to, at least one of an immune disorder or disease, a cardiovascular disorder or disease, an infectious, malignant, and/or neurologic disorder or disease, or other known or specified IL-12/23 related condition.

Such a method can comprise administering an effective amount of a composition or a pharmaceutical composition comprising at least one anti-IL-12/23p40 (or anti-IL-23) antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

### CITATIONS

All publications or patents cited herein, whether or not specifically designated, are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, NY (1987-2001); Sambrook, et al., Molecular Cloning: A Laboratory Manual, 2nd Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

### Antibodies of the Present Invention—Production and Generation

At least one anti-IL-12/23p40 (or anti-IL-23) used in the method of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, N.Y. (1989); Harlow and Lane, antibodies, a Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

A preferred anti-IL-12/23p40 antibody is ustekinumab (Stelara®) having the heavy chain variable region amino acid sequence of SEQ ID NO:7 and the light chain variable region amino acid sequence of SEQ ID NO:8 and having the heavy chain CDR amino acid sequences of SEQ ID NO:1, SEQ ID NO:2, and SEQ ID NO: 3; and the light chain CDR amino acid sequences of SEQ ID NO:4, SEQ ID NO:5, and SEQ ID NO:6. A preferred anti-IL-23 antibody is guselkumab (also referred to as CNTO1959). Other anti-IL-23 antibodies have sequences listed herein and are described in U.S. Pat. No. 7,935,344, the entire contents of which are incorporated herein by reference).

Human antibodies that are specific for human IL-12/23p40 or IL-23 proteins or fragments thereof can be raised against an appropriate immunogenic antigen, such as an isolated IL-12/23p40 protein, IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromyelomas, fusion products thereof, or any cell or fusion cell derived therefrom, or any other suitable cell line as known in the art) (see, e.g., www.atcc.org, www.lifetech.com, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, Calif.; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These non-human amino acid residues are replaced by residues often referred to as “import” residues, which are typically taken from an “import” variable, constant or other domain of a known human sequence.

Known human Ig sequences are disclosed, e.g.,
www.ncbi.nlm.nih.gov/entrez/query.fcgi;
www.ncbi.nih.gov/igblast;
www.atcc.org/phage/hdb.html;
www.mrc-cpe.cam.ac.uk/ALIGNMENTS.php;
www.kabatdatabase.com/top.html;
ftp.ncbi.nih.gov/repository/kabat;
www.sciquest.com;
www.abcam.com;
www.antibodyresource.com/onlinecomp.html;
www.public.iastate.edu/˜pedro/research_tools.html;
www.whfreeman.com/immunology/CH05/kuby05.htm;
www.hhmi.org/grants/lectures/1996/vlab;
www.path.cam.ac.uk/˜mrc7/mikeimages.html;
mcb. harvard.edu/BioLinks/Immunology.html;
www.immunologylink.com;
pathbox.wustl.edu/˜hcenter/index.html;
www.appliedbiosystems.com;
www.nal.usda.gov/awic/pubs/antibody;
www.m.ehime-u.ac.jp/˜yasuhito/Elisa.html;
www.biodesign.com;
www.cancerresearchuk.org;
www.biotech.ufl.edu;
www.isac-net.org; baserv.uci.kun.nl/˜jraats/links1.html;
www.recab.uni-hd.de/immuno.bme.nwu.edu;
www.mrc-cpe.cam.ac.uk;
www.ibt.unam.mx/vir/V_mice.html;
www.bioinf.org.uk/abs; antibody.bath.ac.uk;
www.unizh.ch;
www.cryst.bbk.ac.uk/˜ubcg07s;
www.nimr.mrc.ac.uk/CC/ccaewg/ccaewg.html;
www.path.cam.ac.uk/˜mrc7/humanisation/TAHHP.html;
www.ibt.unam.mx/vir/structure/stat_aim.html;
www.biosci.missouri.edu/smithgp/index.html;
www.jerini.de;
Kabat et al., Sequences of Proteins of Immunological Interest, U.S. Dept. Health (1983),

each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions may be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human anti-IL-12/23p40 (or anti-IL-23) specific antibody used in the method of the present invention may comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human anti-IL-12/23p40 (or anti-IL-23) specific antibody used in the method of the present invention may comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, V14-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively, or additionally, it may be useful to combine amino acid modifications with one or more further amino acid modifications that alter C1q binding and/or the complement dependent cytotoxicity function of the Fc region of an IL-23 binding molecule. The starting polypeptide of particular interest may be one that binds to C1q and displays complement dependent cytotoxicity (CDC). Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human anti-IL-12/23p40 (or anti-IL-23) specific antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of the human anti-IL-12/23p40 (or anti-IL-23) antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human anti-IL-12/23p40 (or anti-IL-23) specific antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation sites that are not present in the polypeptide. Addition of glycosylation sites to the Fc region of a human IL-23 specific antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human anti-IL-12/23p40 (or anti-IL-23) specific antibody of the present invention is expressed in cells that express beta (1,4)-N-acetylglucosaminyltransferase III (GnT III), such that GnT III adds GlcNAc to the human anti-IL-12/23p40 (or anti-IL-23) antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The human anti-IL-12/23p40 (or anti-IL-23) antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL-12/23p40 (or anti-IL-23) antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4) 579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8) 3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL-12/23p40 (or anti-IL-23) antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies used in the method of the present invention can additionally be prepared using at least one anti-IL-12/23p40 (or anti-IL-23) antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies used in the method of the invention can bind human IL-12/IL-23p40 or IL-23 with a wide range of affinities (K_{D}). In a preferred embodiment, a human mAb can optionally bind human IL-12/IL-23p40 or IL-23 with high affinity. For example, a human mAb can bind human IL-12/IL-23p40 or IL-23 with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

### Nucleic Acid Molecules

Using the information provided herein, for example, the nucleotide sequences encoding at least 70-100% of the contiguous amino acids of at least one of the light or heavy chain variable or CDR regions described herein, among other sequences disclosed herein, specified fragments, variants or consensus sequences thereof, or a deposited vector comprising at least one of these sequences, a nucleic acid molecule of the present invention encoding at least one IL-12/IL-23p40 or IL-23 antibody can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules used in the method of the present invention can include nucleic acid molecules comprising an open reading frame (ORF), optionally, with one or more introns, e.g., but not limited to, at least one specified portion of at least one CDR, such as CDR1, CDR2 and/or CDR3 of at least one heavy chain or light chain; nucleic acid molecules comprising the coding sequence for an anti-IL-12/IL-23p40 or IL-23 antibody or variable region; and nucleic acid molecules which comprise a nucleotide sequence substantially different from those described above but which, due to the degeneracy of the genetic code, still encode at least one anti-IL-12/IL-23p40 or IL-23 antibody as described herein and/or as known in the art. Of course, the genetic code is well known in the art. Thus, it would be routine for one skilled in the art to generate such degenerate nucleic acid variants that code for specific anti-IL-12/IL-23p40 or IL-23 antibodies used in the method of the present invention. See, e.g., Ausubel, et al., supra, and such nucleic acid variants are included in the present invention. Non-limiting examples of isolated nucleic acid molecules include nucleic acids encoding HC CDR1, HC CDR2, HC CDR3, LC CDR1, LC CDR2, and LC CDR3, respectively.

As indicated herein, nucleic acid molecules which comprise a nucleic acid encoding an anti-IL-12/IL-23p40 or IL-23 antibody can include, but are not limited to, those encoding the amino acid sequence of an antibody fragment, by itself, the coding sequence for the entire antibody or a portion thereof, the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

### Polynucleotides Selectively Hybridizing to a Polynucleotide as Described Herein

The method of the present invention uses isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides will encode at least a portion of an antibody. The polynucleotides embrace nucleic acid sequences that can be employed for selective hybridization to a polynucleotide encoding an antibody of the present invention. See, e.g., Ausubel, supra; Colligan, supra, each entirely incorporated herein by reference.

### Construction of Nucleic Acids

The isolated nucleic acids can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention, excluding the coding sequence, is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

### Recombinant Methods for Constructing Nucleic Acids

The isolated nucleic acid compositions, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, are well known to those of ordinary skill in the art. (See, e.g., Ausubel, supra; or Sambrook, supra)

### Nucleic Acid Screening and Isolation Methods

A cDNA or genomic library can be screened using a probe based upon the sequence of a polynucleotide used in the method of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent, such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 70-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides used in the method of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

### Synthetic Methods for Constructing Nucleic Acids

The isolated nucleic acids used in the method of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

### Recombinant Expression Cassettes

The present invention uses recombinant expression cassettes comprising a nucleic acid. A nucleic acid sequence, for example, a cDNA or a genomic sequence encoding an antibody used in the method of the present invention, can be used to construct a recombinant expression cassette that can be introduced into at least one desired host cell. A recombinant expression cassette will typically comprise a polynucleotide operably linked to transcriptional initiation regulatory sequences that will direct the transcription of the polynucleotide in the intended host cell. Both heterologous and non-heterologous (i.e., endogenous) promoters can be employed to direct expression of the nucleic acids.

In some embodiments, isolated nucleic acids that serve as promoter, enhancer, or other elements can be introduced in the appropriate position (upstream, downstream or in the intron) of a non-heterologous form of a polynucleotide of the present invention so as to up or down regulate expression of a polynucleotide. For example, endogenous promoters can be altered in vivo or in vitro by mutation, deletion and/or substitution.

### Vectors and Host Cells

The present invention also relates to vectors that include isolated nucleic acid molecules, host cells that are genetically engineered with the recombinant vectors, and the production of at least one anti-IL-23 antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody used in the method of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein used in the method of the present invention. Alternatively, nucleic acids can be expressed in a host cell by turning on (by manipulation) in a host cell that contains endogenous DNA encoding an antibody. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

### Purification of an Antibody

An anti-IL-12/IL-23p40 or IL-23 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

### Anti-IL-12/IL-23p40 or IL-23 Antibodies

An anti-IL-12/IL-23p40 or IL-23 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one C_{H}1, hinge1, hinge2, hinge3, hinge4, C_{H}2, or C_{H}3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

The isolated antibodies used in the method of the present invention comprise the antibody amino acid sequences disclosed herein encoded by any suitable polynucleotide, or any isolated or prepared antibody. Preferably, the human antibody or antigen-binding fragment binds human IL-12/IL-23p40 or IL-23 and, thereby, partially or substantially neutralizes at least one biological activity of the protein. An antibody, or specified portion or variant thereof, that partially or preferably substantially neutralizes at least one biological activity of at least one IL-12/IL-23p40 or IL-23 protein or fragment can bind the protein or fragment and thereby inhibit activities mediated through the binding of IL-12/IL-23p40 or IL-23 to the IL-12 and/or IL-23 receptor or through other IL-12/IL-23p40 or IL-23-dependent or mediated mechanisms. As used herein, the term “neutralizing antibody” refers to an antibody that can inhibit an IL-12/IL-23p40 or IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-12/IL-23p40 or IL-23 antibody to inhibit an IL-12/IL-23p40 or IL-23-dependent activity is preferably assessed by at least one suitable IL-12/IL-23p40 or IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the anti-IL-23 human antibody comprises an IgG1 heavy chain and an IgG1 light chain.

An antibody binds at least one specified epitope specific to at least one IL-12/IL-23p40 or IL-23 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original non-human CDRs can be used. These CDRs may be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The anti-IL-12/IL-23p40 or IL-23 specific antibody can comprise at least one of a heavy or light chain variable region having a defined amino acid sequence. For example, in a preferred embodiment, the anti-IL-12/IL-23p40 or IL-23 antibody comprises an anti-IL-12/IL-23p40 antibody with a heavy chain variable region comprising the amino acid sequence of SEQ ID NO:7 and a light chain variable region comprising the amino acid sequence of SEQ ID NO:8. The anti-IL-12/IL-23p40 or IL-23 specific antibody can also comprise at least one of a heavy or light chain having a defined amino acid sequence. In another preferred embodiment, the anti-IL-12/IL-23p40 or IL-23 antibody comprises an anti-IL-12/IL-23p40 antibody with a heavy chain comprising the amino acid sequence of SEQ ID NO:10 and a light chain comprising the amino acid sequence of SEQ ID NO:11. Antibodies that bind to human IL-12/IL-23p40 or IL-23 and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL-12/IL-23p40 or IL-23 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human IL-12/IL-23p40 or IL-23 with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include, without limitation, replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

### Amino Acid Codes

The amino acids that make up anti-IL-12/IL-23p40 or IL-23 antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE | THREE | | THREE |
|---|---|---|---|
| LETTER | LETTER | | NUCLEOTIDE |
| CODE | CODE | NAME | CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, |
| | | | CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, |
| | | | CGG, CGU |
| S | Ser | Serine | AGC, AGU, UCA, UCC, |
| | | | UCG, UCU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

### Sequences

### Example Anti-IL-12/IL-23p40 Antibody Sequences—STELARA® (Ustekinumab)

Amino acid sequence of anti-IL-12/IL-23p40 antibody complementarity determining region heavy chain 1 (CDRH1): (SEQ ID NO:1)

TYWLG

Amino acid sequence of anti-IL-12/IL-23p40 antibody complementarity determining region heavy chain 2 (CDRH2): (SEQ ID NO:2)

IMSPVDSDIRYSPSFQG

Amino acid sequence of anti-IL-12/IL-23p40 antibody complementarity determining region heavy chain 3 (CDRH3): (SEQ ID NO:3)

RRPGQGYFDF

Amino acid sequence of anti-IL-12/IL-23p40 antibody complementarity determining region light chain 1 (CDRL1): (SEQ ID NO:4)

RASQGISSWLA

Amino acid sequence of anti-IL-12/IL-23p40 antibody complementarity determining region light chain 2 (CDRL2): (SEQ ID NO:5)

AASSLQS

Amino acid sequence of anti-IL-12/IL-23p40 antibody complementarity determining region light chain 3 (CDRL3): (SEQ ID NO:6)

QQYNIYPYT

Amino acid sequence of anti-IL-12/IL-23p40 antibody variable heavy chain region (CDRs underlined): (SEQ ID NO:7)

| | |
|---|---|
| 1 | EVQLVQSGAE VKKPGESLKI SCKGSGYSFT TYWLGWVRQM PGKGLDWIGI MSPVDSDIRY |
| 61 | SPSFQGQVTM SVDKSITTAY LQWNSLKASD TAMYYCARRR PGQGYFDFWG QGTLVTVSS |

Amino acid sequence of anti-IL-12/IL-23p40 antibody variable light chain region (CDRs underlined): (SEQ ID NO:8)

| | |
|---|---|
| 1 | DIQMTQSPSS LSASVGDRVT ITCRASQGIS SWLAWYQQKP EKAPKSLIYA ASSLQSGVPS |
| 61 | RFSGSGSGTD FTLTISSLQP EDFATYYCQQ YNIYPYTFGQ GTKLEIKR |

Amino acid sequence of anti-IL-12/IL-23p40 antibody heavy chain (CDRs underlined): (SEQ ID NO:10)

| | |
|---|---|
| 1 | EVQLVQSGAE VKKPGESLKI SCKGSGYSFT TYWLGWVRQM PGKGLDWIGI MSPVDSDIRY |
| 61 | SPSFQGQVTM SVDKSITTAY LQWNSLKASD TAMYYCARRR PGQGYFDFWG QGTLVTVSSS |
| 121 | STKGPSVFPL APSSKSTSGG TAALGCLVKD YFPEPVTVSW NSGALTSGVH TFPAVLQSSG |
| 181 | LYSLSSVVTV PSSSLGTQTY ICNVNHKPSN TKVDKRVEPK SCDKTHTCPP CPAPELLGGP |
| 241 | SVFLFPPKPK DTLMISRTPE VTCVVVDVSH EDPEVKFNWY VDGVEVHNAK TKPREEQYNS |
| 301 | TYRVVSVLTV LHQDWLNGKE YKCKVSNKAL PAPIEKTISK AKGQPREPQV YTLPPSRDEL |
| 361 | TKNQVSLTCL VKGFYPSDIA VEWESNGQPE NNYKTTPPVL DSDGSFFLYS KLTVDKSRWQ |
| 421 | QGNVFSCSVM HEALHNHYTQ KSLSLSPGK |

Amino acid sequence of anti-IL-12/IL-23p40 antibody light chain (CDRs underlined): (SEQ ID NO:11)

| | |
|---|---|
| 1 | DIQMTQSPSS LSASVGDRVT ITCRASQGIS SWLAWYQQKP EKAPKSLIYA ASSLQSGVPS |
| 61 | RFSGSGSGTD FTLTISSLQP EDFATYYCQQ YNIYPYTFGQ GTKLEIKRTV AAPSVFIFPP |
| 121 | SDEQLKSGTA SVVCLLNNFY PREAKVQWKV DNALQSGNSQ ESVTEQDSKD STYSLSSTLT |
| 181 | LSKADYEKHK VYACEVTHQG LSSPVTKSFN RGEC |

### Amino Acid Sequence IL-12

Amino acid sequence of human interleukin (IL)-12 with alpha and beta subunits: (SEQ ID NO:9)

| | |
|---|---|
| 1 | RNLPVATPDP GMFPCLHHSQ NLLRAVSNML QKARQTLEFY PCTSEEIDHE DITKDKTSTV |
| 61 | EACLPLELTK NESCLNSRET SFITNGSCLA SRKTSFMMAL CLSSIYEDLK MYQVEFKTMN |
| 121 | AKLLMDPKRQ IFLDQNMLAV IDELMQALNF NSETVPQKSS LEEPDFYKTK IKLCILLHAF |
| 181 | RIRAVTIDRV MSYLNASIWE LKKDVYVVEL DWYPDAPGEM VVLTCDTPEE DGITWTLDQS |
| 241 | SEVLGSGKTL TIQVKEFGDA GQYTCHKGGE VLSHSLLLLH KKEDGIWSTD ILKDQKEPKN |
| 301 | KTFLRCEAKN YSGRFTCWWL TTISTDLTFS VKSSRGSSDP QGVTCGAATL SAERVRGDNK |
| 361 | EYEYSVECQE DSACPAAEES LPIEVMVDAV HKLKYENYTS SFFIRDIIKP DPPKNLQLKP |
| 421 | LKNSRQVEVS WEYPDTWSTP HSYFSLTFCV QVQGKSKREK KDRVFTDKTS ATVICRKNAS |
| 481 | ISVRAQDRYY SSSWSEWASV PCS |

An anti-IL-12/IL-23p40 or IL-23 antibody used in the method of the present invention can include one or more amino acid substitutions, deletions or additions, either from natural mutations or human manipulation, as specified herein.

The number of amino acid substitutions a skilled artisan would make depends on many factors, including those described above. Generally speaking, the number of amino acid substitutions, insertions or deletions for any given anti-IL-12/IL-23p40 or IL-23 antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-IL-12/IL-23p40 or IL-23 specific antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL-12/IL-23p40 or IL-23 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Anti-IL-12/IL-23p40 or IL-23 antibodies can include, but are not limited to, at least one portion, sequence or combination selected from 5 to all of the contiguous amino acids of at least one of SEQ ID NOs 1, 2, 3, 4, 5, 6, 7, 8, 10, or 11.

IL-12/IL-23p40 or IL-23 antibodies or specified portions or variants can include, but are not limited to, at least one portion, sequence or combination selected from at least 3-5 contiguous amino acids of the SEQ ID NOs above; 5-17 contiguous amino acids of the SEQ ID NOs above, 5-10 contiguous amino acids of the SEQ ID NOs above, 5-11 contiguous amino acids of the SEQ ID NOs above, 5-7 contiguous amino acids of the SEQ ID NOs above; 5-9 contiguous amino acids of the SEQ ID NOs above.

An anti-IL-12/IL-23p40 or IL-23 antibody can further optionally comprise a polypeptide of at least one of 70-100% of 5, 17, 10, 11, 7, 9, 119, 108, 449, or 214 contiguous amino acids of the SEQ ID NOs above. In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of the SEQ ID NOs above. For example, the amino acid sequence of a light chain variable region can be compared with the sequence of the SEQ ID NOs above, or the amino acid sequence of a heavy chain CDR3 can be compared with the SEQ ID NOs above. Preferably, 70-100% amino acid identity (i.e., 90, 91, 92, 93, 94, 95, 96, 97, 98, 99, 100 or any range or value therein) is determined using a suitable computer algorithm, as known in the art.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

Preferred parameters for polypeptide sequence comparison include the following:
(1) Algorithm: Needleman and Wunsch, J. Mol Biol. 48:443-453 (1970) Comparison matrix: BLOSSUM62 from Hentikoff and Hentikoff, Proc. Natl. Acad. Sci, USA. 89:10915-10919 (1992)
Gap Penalty: 12
Gap Length Penalty: 4
A program useful with these parameters is publicly available as the “gap” program from Genetics Computer Group, Madison Wis. The aforementioned parameters are the default parameters for peptide sequence comparisons (along with no penalty for end gaps).

Preferred parameters for polynucleotide comparison include the following:
(1) Algorithm: Needleman and Wunsch, J. Mol Biol. 48:443-453 (1970)
Comparison matrix: matches=+10, mismatch=0
Gap Penalty: 50
Gap Length Penalty: 3
Available as: The “gap” program from Genetics Computer Group, Madison Wis. These are the default parameters for nucleic acid sequence comparisons.

Byway of example, a polynucleotide sequence may be identical to another sequence, that is 100% identical, or it may include up to a certain integer number of nucleotide alterations as compared to the reference sequence. Such alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the sequence by the numerical percent of the respective percent identity (divided by 100) and subtracting that product from the total number of nucleotides in the sequence, or:

n.sub.n.ltorsim.x.sub.n-(x.sub.n.y),
wherein n.sub.n is the number of nucleotide alterations, x.sub.n is the total number of nucleotides in sequence, and y is, for instance, 0.70 for 70%, 0.80 for 80%, 0.85 for 85%, 0.90 for 90%, 0.95 for 95%, etc., and wherein any non-integer product of x.sub.n and y is rounded down to the nearest integer prior to subtracting from x.sub.n.

Alterations of a polynucleotide sequence encoding the SEQ ID NOs above may create nonsense, missense or frameshift mutations in this coding sequence and thereby alter the polypeptide encoded by the polynucleotide following such alterations. Similarly, a polypeptide sequence may be identical to the reference sequence of the SEQ ID NOs above, that is be 100% identical, or it may include up to a certain integer number of amino acid alterations as compared to the reference sequence such that the percentage identity is less than 100%. Such alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the SEQ ID NOs above by the numerical percent of the respective percent identity (divided by 100) and then subtracting that product from the total number of amino acids in the SEQ ID NOs above, or: n.sub.a.ltorsim.x.sub.a-(x.sub.a.y), wherein n.sub.a is the number of amino acid alterations, x.sub.a is the total number of amino acids in the SEQ ID NOs above, and y is, for instance 0.70 for 70%, 0.80 for 80%, 0.85 for 85% etc., and wherein any non-integer produce of x.sub.a and y is rounded down to the nearest integer prior to subtracting it from x.sub.a.

Exemplary heavy chain and light chain variable regions sequences and portions thereof are provided in the SEQ ID NOs above. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL-12/IL-23p40 or IL-23 antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and, preferably, at least 50%, 60%, or 70%, and, most preferably, at least 80%, 90%, or 95%-100% or more (including, without limitation, up to 10 times the specific activity) of that of the native (non-synthetic), endogenous or related and known antibody. Methods of assaying and quantifying measures of enzymatic activity and substrate specificity are well known to those of skill in the art.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG₅₀₀₀ and PEG_{20,000}, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C₁₂, laurate), n-tetradecanoate (C14, myristate), n-octadecanoate (Cis, stearate), n-eicosanoate (C₂₀, arachidate), n-docosanoate (C₂₂, behenate), n-triacontanoate (C₃₀), n-tetracontanoate (C₄₀), cis-Δ9-octadecanoate (Cis, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

The method of the present invention also uses an anti-IL-12/IL-23p40 or IL-23 antibody composition comprising at least one, at least two, at least three, at least four, at least five, at least six or more anti-IL-12/IL-23p40 or IL-23 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL-12/IL-23p40 or IL-23 antibody amino acid sequence selected from the group consisting of 70-100% of the contiguous amino acids of the SEQ ID NOs above, or specified fragments, domains or variants thereof. Preferred anti-IL-12/IL-23p40 or IL-23 antibody compositions include at least one or two full length, fragments, domains or variants as at least one CDR or LBP containing portions of the anti-IL-12/IL-23p40 or IL-23 antibody sequence described herein, for example, 70-100% of the SEQ ID NOs above, or specified fragments, domains or variants thereof. Further preferred compositions comprise, for example, 40-99% of at least one of 70-100% of the SEQ ID NOs above, etc., or specified fragments, domains or variants thereof. Such composition percentages are by weight, volume, concentration, molarity, or molality as liquid or dry solutions, mixtures, suspension, emulsions, particles, powder, or colloids, as known in the art or as described herein.

### Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21^{st} edition, Springhouse Corp., Springhouse, P A, 2001; Health Professional's Drug Guide 2001, ed., Shannon, Wilson, Stang, Prentice-Hall, Inc, Upper Saddle River, N.J.; Pharmacotherapy Handbook, Wells et al., ed., Appleton & Lange, Stamford, Conn., each entirely incorporated herein by reference).

Byway of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one coricosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, 6-mercaptopurine, methotrexate, mizoribine, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (See, e.g., pp. 1098-1136 of Nursing 2001 Drug Handbook.)

Anti-IL-12/IL-23p40 or IL-23 antibody compositions can further comprise at least one of any suitable and effective amount of a composition or pharmaceutical composition comprising at least one anti-IL-12/IL-23p40 or IL-23 antibody contacted or administered to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., basiliximab, cyclosporine, daclizumab), a cytokine or a cytokine antagonist. Non-limiting examples of such cytokines include, but are not limited to, any of IL-1 to IL-23 et al. (e.g., IL-1, IL-2, etc.). Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

Anti-IL-12/IL-23p40 or IL-23 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the anti-IL-23 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-12/IL-23p40 or IL-23 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-12/IL-23p40 or IL-23 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-12/IL-23p40 or IL-23 antibody, portion or variant compositions according to the invention are known in the art, e.g., as listed in “Remington: The Science & Practice of Pharmacy,” 19^{th} ed., Williams & Williams, (1995), and in the “Physician's Desk Reference,” 52^{nd} ed., Medical Economics, Montvale, N.J. (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

### Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-12/IL-23p40 or IL-23 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL-12/IL-23p40 or IL-23 antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL-12/IL-23p40 or IL-23 antibody, and a second vial comprising an aqueous diluent of prescribed buffer or preservative, wherein said packaging material comprises a label that instructs a patient to reconstitute the anti-IL-12/IL-23p40 or IL-23 antibody in the aqueous diluent to form a solution that can be held over a period of twenty-four hours or greater.

The anti-IL-12/IL-23p40 or IL-23 antibody used in accordance with the present invention can be produced by recombinant means, including from mammalian cell or transgenic preparations, or can be purified from other biological sources, as described herein or as known in the art.

The range of the anti-IL-12/IL-23p40 or IL-23 antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), PLURONIC® (polymer) F68 (polyoxyethylene polyoxypropylene block copolymers), and PEG (polyethylene glycol) or non-ionic surfactants, such as polysorbate 20 or 80 or poloxamer 184 or 188, PLURONIC® (polymer), e.g., polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL-12/IL-23p40 or IL-23 antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-12/IL-23p40 or IL-23 specific antibody and preservative in an aqueous diluent is carried out using conventional dissolution and mixing procedures. To prepare a suitable formulation, for example, a measured amount of at least one anti-IL-12/IL-23p40 or IL-23 antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to patients as clear solutions or as dual vials comprising a vial of lyophilized anti-IL-12/IL-23p40 or IL-23 specific antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL-12/IL-23p40 or IL-23 specific antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-IL-12/IL-23p40 or IL-23 specific antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-IL-12/IL-23p40 or IL-23 specific antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising these single vial systems include those pen-injector devices for delivery of a solution such as B-D® (pen injector device), NOVOPEN® (pen injector device), AUTOPEN® (pen injector device), OPTIPEN® (pen injector device), GENOTROPIN PEN® (pen injector device), BIOJECTOR® (pen injector device), Reco-Pen, Humaject, J-tip Needle-Free Injector, Intraject, Medi-Ject, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com). Recognized devices comprising a dual vial system include those pen-injector systems for reconstituting a lyophilized drug in a cartridge for delivery of the reconstituted solution such as the HUMATROPEN® (pen injector device).

The products may include packaging material. The packaging material provides, in addition to the information required by the regulatory agencies, the conditions under which the product can be used. The packaging material of the present invention provides instructions to the patient, as applicable, to reconstitute the at least one anti-IL-12/IL-23p40 or IL-23 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL-12/IL-23p40 or IL-23 antibody and a selected buffer, preferably, a phosphate buffer containing saline or a chosen salt. Mixing the anti-IL-23 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal patient. Such pharmaceutical compositions are prepared using water at “standard state” as the diluent and routine methods well known to those of ordinary skill in the art. For example, buffering components such as histidine and histidine monohydrochloride hydrate, may be provided first followed by the addition of an appropriate, non-final volume of water diluent, sucrose and polysorbate 80 at “standard state.” Isolated antibody may then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions may be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g. 25° C.+/−2° C. and a pressure of 1 atmosphere).

Importantly, such pharmaceutical compositions may contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL-12/IL-23p40 or IL-23 specific mAbs bind to similar or different epitopes and/or compete with each other. Abs are individually coated on ELISA plates. Competing mAbs are added, followed by the addition of biotinylated hrIL-12 or IL-23. For positive control, the same mAb for coating may be used as the competing mAb (“self-competition”). IL-12/IL-23p40 or IL-23 binding is detected using streptavidin. These results demonstrate whether the mAbs recognize similar or partially overlapping epitopes on IL-12/IL-23p40 or IL-23.

One aspect of the method of the invention administers to a patient a pharmaceutical composition comprising

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to patients as clear solutions or as dual vials comprising a vial of lyophilized at least one anti-IL-23 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-23 antibody may result in other than a clear solution of lyophilized powder comprising the antibody. Among non-clear solutions are formulations comprising particulate suspensions, said particulates being a composition containing the anti-IL-23 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gelatin agar, starch, arabinogalactan, albumin, collagen, polyglycolic acid, polylactic aced, glycolide-L(-) lactide poly(epsilon-caprolactone, poly(epsilon-caprolactone-CO-lactic acid), poly(epsilon-caprolactone-CO-glycolic acid), poly(ß-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(-) lactide poly(epsilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

An anti-IL-23 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

### Therapeutic Applications

The present invention also provides a method for modulating or treating lupus, in a cell, tissue, organ, animal, or patient, as known in the art or as described herein, using at least one IL-23 antibody of the present invention, e.g., administering or contacting the cell, tissue, organ, animal, or patient with a therapeutic effective amount of IL-12/IL-23p40 or IL-23 specific antibody.

Any method of the present invention can comprise administering an effective amount of a composition or pharmaceutical composition comprising an anti-IL-23 antibody to a cell, tissue, organ, animal or patient in need of such modulation, treatment or therapy. Such a method can optionally further comprise co-administration or combination therapy for treating such diseases or disorders, wherein the administering of said at least one anti-IL-23 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, C A (2000); Nursing 2001 Handbook of Drugs, 21^{st} edition, Springhouse Corp., Springhouse, P A, 2001; Health Professional's Drug Guide 2001, ed., Shannon, Wilson, Stang, Prentice-Hall, Inc, Upper Saddle River, N.J., each of which references are entirely incorporated herein by reference.

### Therapeutic Treatments

Typically, treatment of lupus is affected by administering an effective amount or dosage of an anti-IL-12/23p40 or anti-IL-23 antibody composition that total, on average, a range from at least about 0.01 to 500 milligrams of an anti-IL-12/23p40 or anti-IL-23 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and, preferably, 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions, the active ingredient will ordinarily be present in an amount of about 0.5-99.999% by weight based on the total weight of the composition.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Alternative Administration

Many known and developed modes can be used according to the present invention for administering pharmaceutically effective amounts of an anti-IL-23 antibody. While pulmonary administration is used in the following description, other modes of administration can be used according to the present invention with suitable results. IL-12/IL-23p40 or IL-23 antibodies of the present invention can be delivered in a carrier, as a solution, emulsion, colloid, or suspension, or as a dry powder, using any of a variety of devices and methods suitable for administration by inhalation or other modes described here within or known in the art.

### Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference. Alternative Delivery

The invention further relates to the administration of an anti-IL-12/IL-23p40 or IL-23 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-12/IL-23p40 or IL-23 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### Example: Manufacturing Processes to Produce STELARA® (Ustekinumab)

### Background

STELARA® (ustekinumab) is a fully human G1 kappa monoclonal antibody that binds with high affinity and specificity to the shared p40 subunit of human interleukin (IL)-12 and IL-23 cytokines. Ustekinumab comprises a heavy chain of the amino acid sequence of SEQ ID NO:10 and a light chain of the amino acid sequence of SEQ ID NO:11; a heavy chain variable domain amino acid sequence of SEQ ID NO:7; and a light chain variable domain amino acid sequence of SEQ ID NO: 8; the heavy chain CDR amino acid sequences of SEQ ID NO:1, SEQ ID NO:2, and SEQ ID NO:3; and the light chain CDR amino acid sequences of SEQ ID NO:4, SEQ ID NO:5, and SEQ ID NO:6. The binding of ustekinumab to the IL-12/23p40 subunit blocks the binding of IL-12 or IL-23 to the IL-12Rβ1 receptor on the surface of natural killer and CD4⁺ T cells, inhibiting IL-12- and IL-23-specific intracellular signaling and subsequent activation and cytokine production. Abnormal regulation of IL-12 and IL-23 has been associated with multiple immune-mediated diseases.

To date, ustekinumab has received marketing approval globally, including countries in North America, Europe, South America, and the Asia-Pacific region, for the treatment of adult patients including those with chronic moderate to severe plaque psoriasis and/or active psoriatic arthritis. Ustekinumab is also being evaluated in a Phase 3 studies for Crohn's disease (CD) and in a proof of concept study for the treatment of active Systemic Lupus Erythematosus (SLE).

### Manufacturing Process Overview

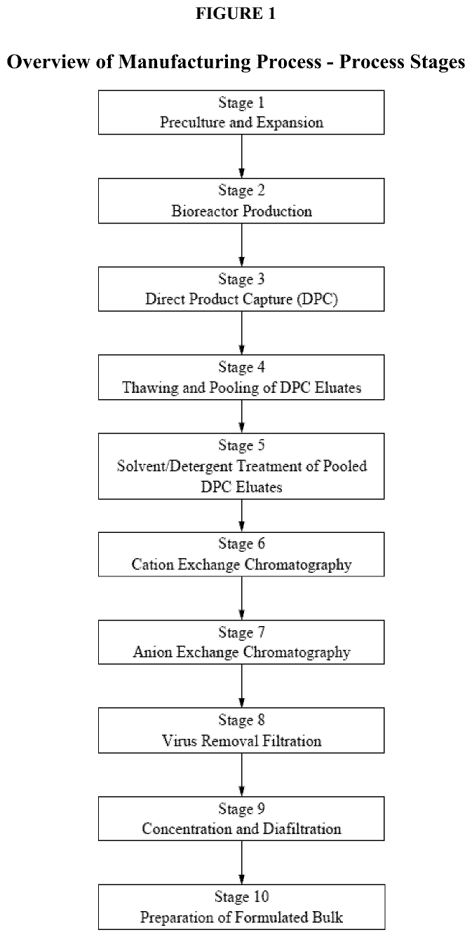

STELARA® (ustekinumab) is manufactured in a 10-stage process that includes continuous perfusion cell culture followed by purification. An overview of the manufacturing process is provided in FIG. 1.

As used herein, the terms “culture”, “culturing”, “cultured”, and “cell culture” refer to a cell population that is suspended in a medium under conditions suitable to survival and/or growth of the cell population. As will be clear from context to those of ordinary skill in the art, these terms as used herein also refer to the combination comprising the cell population and the medium in which the population is suspended. Cell culture includes, e.g., cells grown by batch, fed-batch or perfusion cell culture methods and the like. In certain embodiments, the cell culture is a mammalian cell culture.

Cell lines for use in the present invention include mammalian cell lines including, but not limited to, Chinese hamster vary cells (CHO cells), human embryonic kidney cells (HEK cells), baby hamster kidney cells (BHK cells), mouse myeloma cells (e.g., NS0 cells and Sp2/0 cells), and human retinal cells (e.g., PER.C6 cells).

As used herein, the terms “chemically defined medium”, “chemically defined media”, “chemically defined hybridoma medium”, or “chemically defined hybridoma media” refer to a synthetic growth medium in which the identity and concentration of all the components are known. Chemically defined media do not contain bacterial, yeast, animal, or plant extracts, animal serum or plasma although they may or may not include individual plant or animal-derived components (e.g., proteins, polypeptides, etc). Chemically defined media may contain inorganic salts such as phosphates, sulfates, and the like needed to support growth. The carbon source is defined, and is usually a sugar such as glucose, lactose, galactose, and the like, or other compounds such as glycerol, lactate, acetate, and the like. While certain chemically defined media also use phosphate salts as a buffer, other buffers may be employed such as citrate, triethanolamine, and the like. Examples of commercially available chemically defined media include, but are not limited to, ThermoFisher's CD Hybridoma Medium and CD Hybridoma AGT™ Medium, various Dulbecco's Modified Eagle's (DME) mediums (Sigma-Aldrich Co; SAFC Biosciences, Inc), Ham's Nutrient Mixture (Sigma-Aldrich Co; SAFC Biosciences, Inc), combinations thereof, and the like. Methods of preparing chemically defined mediums are known in the art, for example in U.S. Pat. Nos. 6,171,825 and 6,936,441, WO 2007/077217, and U.S. Patent Application Publication Nos. 2008/0009040 and 2007/0212770.

The term “bioreactor” as used herein refers to any vessel useful for the growth of a cell culture. The bioreactor can be of any size so long as it is useful for the culturing of cells. In certain embodiments, such cells are mammalian cells. Typically, the bioreactor will be at least 1 liter and may be 10, 100, 250, 500, 1,000, 2,500, 5,000, 8,000, 10,000, 12,000 liters or more, or any volume in between. The internal conditions of the bioreactor, including, but not limited to pH and temperature, are optionally controlled during the culturing period. The bioreactor can be composed of any material that is suitable for holding mammalian cell cultures suspended in media under the culture conditions of the present invention, including glass, plastic or metal. The term “production bioreactor” as used herein refers to the final bioreactor used in the production of the polypeptide or glycoprotein of interest. The volume of the production bioreactor is typically at least 500 liters and may be 1,000, 2,500, 5,000, 8,000, 10,000, 12,000 liters or more, or any volume in between. One of ordinary skill in the art will be aware of and will be able to choose suitable bioreactors for use in practicing the present invention.

Preculture, expansion, and production of ustekinumab are performed in Stage 1 and Stage 2. In Stage 1, preculture is initiated from one or more working cell bank vials of transfected Sp2/0 cells expressing the HC and LC sequences of ustekinumab and expanded in culture flasks, disposable culture bags, and a 100 L seed bioreactor. The cells are cultured until the cell density and volume required for inoculation of a 500 L production bioreactor are obtained. In Stage 2, the cell culture is perfused in a 500 L production bioreactor using an alternating tangential flow (ATF) hollow fiber filter cell retention system. Cell culture permeate (harvest) is collected from the ATF system while cells are retained within the bioreactor and the culture is replenished with fresh medium. Harvest from one or more 500 L production bioreactors may be combined in Stage 3. The harvests are purified using MabSelect Protein A resin affinity chromatography. The resultant direct product capture (DPC) eluate is frozen until further processing.

Purification of ustekinumab from DPC is performed in Stage 4 through Stage 8 by ion exchange chromatography steps and other steps to inactivate or remove potential virus contamination (solvent/detergent [S/D] treatment and virus removal filtration). DPC eluates are thawed, pooled and filtered in Stage 4 and incubated with Tri-n-butyl Phosphate (TNBP) and polysorbate 80 S/D treatment in Stage 5 to inactivate any lipid-enveloped viruses present. TNBP and polysorbate 80 reagents, aggregates, and impurities are removed from ustekinumab in Stage 6, using SPXL SEPAROSE® (resin) cation exchange chromatography. Ustekinumab is further purified using QXL SEPAROSE® (resin) anion exchange chromatography in Stage 7 to remove DNA, viruses, and impurities. SPXL and QXL resins obtained from GE Healthcare Bio-Sciences, Pittsburgh, Pa. In Stage 8, the purified ustekinumab is diluted and filtered through an NFP virus retentive filter (Millipore Sigma, Burlington, Mass.).

Preparation of the ustekinumab pre-formulated bulk (PFB) and formulated bulk (FB) is performed in Stages 9 and 10, respectively. In Stage 9, the ultrafiltration step concentrates the ustekinumab and the diafiltration step adds the formulation excipients and removes the in-process buffer salts. Polysorbate 80 is added to the ustekinumab PFB in Stage 10 to obtain the FB. The FB is filtered into polycarbonate containers for frozen storage. The frozen FB is packaged in insulated containers with dry ice for transport to the drug product manufacturing site.

### Detailed Description of Cell Culture in Manufacturing Process

### Stage 1

### Preculture and Expansion

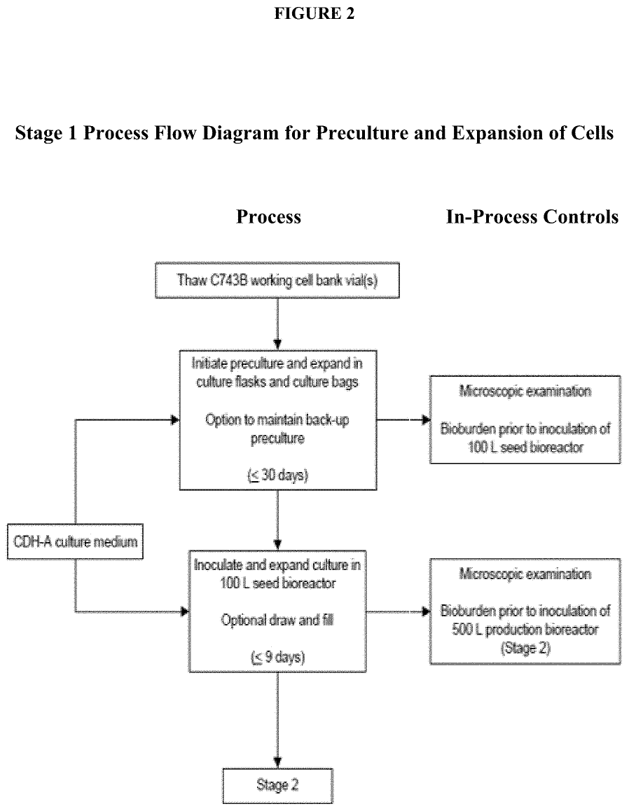

The first stage in the production of ustekinumab is the initiation of preculture from a Working Cell Bank (WCB) vial of transfected Sp2/0 cells expressing the HC and LC sequences of ustekinumab and expanded in culture flasks, disposable culture bags, and a 100 L seed bioreactor. The cells are cultured until the cell density and volume required for inoculation of a 500 L production bioreactor are obtained. A flow diagram depicting the preculture and expansion process is provided in FIG. 2.

### Manufacturing Procedure

One or more cryopreserved vials of WCB are thawed and diluted with CD (chemically defined) hybridoma medium supplemented with 6 mM L-glutamine, 0.5 mg/L mycophenolic acid, 2.5 mg/L hypoxanthine, and 50 mg/L xanthine (CDH-A). The culture viability must be ≥45%. The cells are further diluted with CDH-A in a culture flask to a seeding density of 0.2 to 0.5×10⁶ viable cells (VC)/mL. The preculture is maintained in a humidified CO2 incubator, with temperature, CO2 concentration, and agitation controlled within ranges defined in the batch record. The preculture is incubated for 3 days until a minimum cell density of ≥0.6×10⁶ VC/mL and a culture viability of ≥50% are obtained. The preculture is expanded sequentially in a series of culture flasks and then culture bags as a mechanism to scale up for inoculation of the 100 L seed bioreactor. During the culture expansion phase, each incubation step takes 3 days to achieve passage conditions, which require a cell density of ≥0.6×10⁶ VC/mL and a culture viability of ≥80%. The seeding density for each passage is 0.2 to 0.5×10⁶ VC/mL in culture flasks, and 0.2 to 0.6×10⁶ VC/mL in culture bags. Each passage is sampled for viable cell density (VCD), culture viability, and microscopic examination. Prior to inoculation of the 100 L seed bioreactor, the preculture is sampled for bioburden.

Preculture expansions may be maintained for a maximum of 30 days post-thaw. Precultures not used within 30 days are discarded. Back-up precultures, expanded as described above and subject to the same in-process monitoring, control tests, and process parameters as the primary precultures, may be maintained and used to inoculate another 100 L seed bioreactor as needed

When the preculture meets inoculum criteria, the contents of the culture bag(s) are transferred to the 100 L seed bioreactor containing CDH-A to target a seeding density of ≥0.3×10⁶ VC/mL. The seed bioreactor culture pH, temperature, and dissolved oxygen concentration are controlled within ranges defined in the batch record. The culture is expanded until a cell density of ≥1.5×10⁶ VC/mL and a culture viability of ≥80% are obtained. The culture is sampled for VCD, culture viability, and microscopic examination throughout the seed bioreactor process. Prior to inoculation of the 500 L production bioreactor, the culture is sampled for bioburden.

When the VCD of the seed bioreactor culture reaches ≥1.5×10⁶ VC/mL, the culture may be used to inoculate the 500 L production bioreactor. Alternatively, a portion of the culture can be drawn from the 100 L seed bioreactor and the remaining culture diluted with fresh medium. Following this “draw and fill” process, the culture is allowed to expand to sufficient cell density to inoculate the 500 L production bioreactor. The maximum duration of the 100 L seed bioreactor culture is 9 days post-inoculation.

### Stage 2

### Bioreactor Production

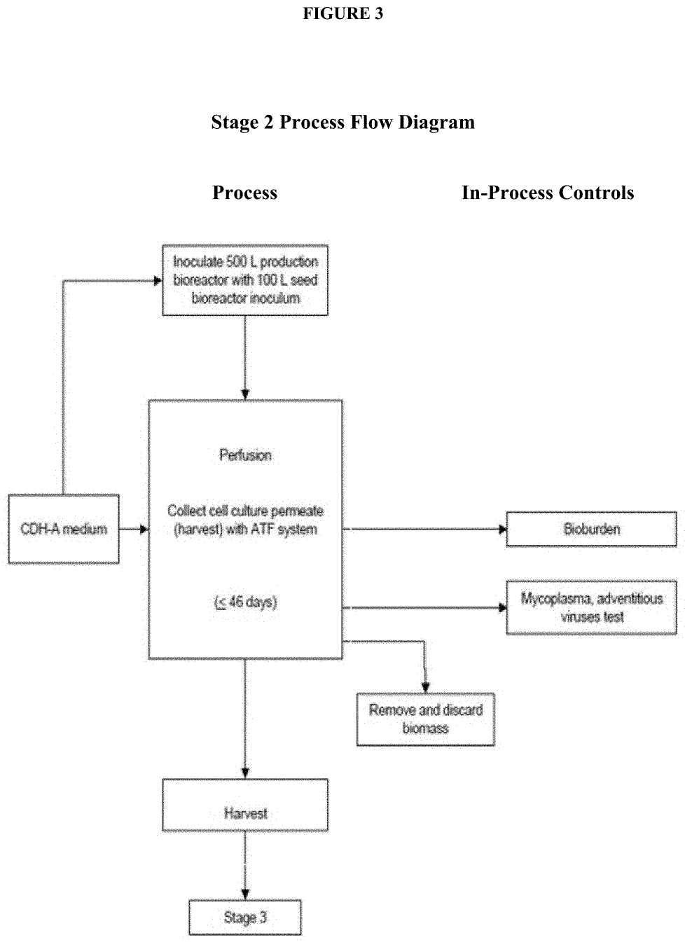

In Stage 2, cell culture is continuously perfused in a 500 L production bioreactor using an alternating tangential flow hollow fiber filter cell retention system (ATF system). Cell culture permeate (harvest) is collected from the ATF system while cells are returned to the bioreactor, and the culture is replenished with fresh medium. A flow diagram depicting the bioreactor production process is provided in FIG. 3.

### Manufacturing Procedure

The inoculation of the 500 L production bioreactor is performed by transferring the contents of the 100 L seed bioreactor into the 500 L production bioreactor containing CD (chemically defined) hybridoma medium supplemented with 6 mM L-glutamine, 0.5 mg/L mycophenolic acid, 2.5 mg/L hypoxanthine, and 50 mg/L xanthine (CDH-A). The volume transferred must be sufficient to target a seeding density of ≥0.3×10⁶ viable cells (VC)/mL. The culture is maintained at a temperature of 34 to 38° C., a pH of 6.8 to 7.6, and a dissolved oxygen (DO) concentration of 1 to 100%.

Continuous perfusion is initiated, and culture is drawn from the 500 L bioreactor into the ATF system to separate the cells from the permeate. The permeate is filtered through the 0.2 μm ATF filter and collected as harvest in bioprocess containers (BPCs). The cells are returned to the bioreactor, and fresh CDH-A is supplied to maintain a constant culture volume. Viable cell density (VCD), culture viability, pH, DO, temperature and immunoglobulin G (IgG) content are monitored during the production run. The perfusion rate is gradually increased in proportion to VCD until a target rate of approximately one bioreactor volume per day is reached. The perfusion rate is controlled, not to exceed 1.20 bioreactor volumes per day. Retention of the ATF system is monitored to facilitate shutdown of an ATF filter prior to the IgG retention across the filter exceeding 50%.

When the VCD within the 500 L bioreactor reaches 8.0×10⁶ VC/mL or on day 10, whichever comes first, the pH target is lowered from 7.2 to 7.1. Biomass removal is initiated at either day 20 or when a VCD of 12.0×10⁶ VC/mL is reached, whichever comes first. Biomass is removed from the 500 L production bioreactor into BPCs at a rate of up to 20% bioreactor volumes per day. Each harvest is sampled for bioburden.

The continuous perfusion cell culture operation in the 500 L production bioreactor continues for up to 46 days post-inoculation. At the end of production, the culture is sampled for mycoplasma and adventitious virus testing. Harvest may be stored for ≤30 days at 2 to 8° C. after disconnection from the bioreactor

### Introduction to Manufacturing Control Strategy

A manufacturing control strategy was developed to maintain consistent drug substance (DS) and drug product (DP) characteristics of ustekinumab with regard to oligosaccharide profile and also to control viable cell viability and productivity during large-scale commercial production. Ustekinumab glycosylation is monitored for formulated bulk (FB) at Stage 10 of the manufacturing process, with upper and lower specifications in place for peak 3 area % in the cIEF profile, % total neutral oligosaccharides, % total charged oligosaccharides, and % individual neutral oligosaccharide species, including, G0F, G1F, and G2F. As used herein, the terms “drug substance” (abbreviated as “DS”) and “drug product” (abbreviated as “DP”) refer to a composition or compositions for use as commercial drugs, for example in clinical trials or as marketed drugs. A DS is an active ingredient that is intended to furnish pharmacological activity or other direct effect in the diagnosis, cure, mitigation, treatment, or prevention of disease or to affect the structure or any function of the human body. The formulated bulk (FB) produced in the manufacturing process is the drug substance (DS). A DP (also referred to as a medicinal product, medicine, medication, or medicament) is a drug used in the diagnosis, cure, mitigation, treatment, or prevention of disease or to affect the structure or any function of the human body. The DP is the DS that has been prepared as the medicinal product for sale and/or administration to the patient. As used herein, the terms “manufacturing control strategy”, “manufacturing strategy”, “control strategy”, and “method of manufacture”, refer to processes for producing the DS or DP for commercial use, for example in clinical trials or as marketed drugs.

In brief, the manufacturing control strategy ensures that the oligosaccharide profile of ustekinumab is controlled by culturing cells in chemically defined media that is controlled to contain specified trace metal concentrations consisting of, Mn²⁺ (manganese) ≥10.0 μg/liter to 35.0 μg/liter and Cu²⁺ (copper) ≥1.0 μg/liter to ≤1.8 μg/liter. As used herein, the term “specified” refers to identifying clearly and definitely that the concentrations of manganese and copper are requirements and must be precisely controlled within upper and lower limits in the chemically defined media. As used herein the term “controlled” refers to carefully regulating, testing and verifying, e.g., carefully weighing and/or measuring by other means the raw materials containing manganese and copper during production of the media, measuring the final concentrations of manganese and copper in the chemically defined media using inductively coupled plasma mass spectrometry (IPC-MS) or other methods and if necessary adjusting the concentrations by supplementing the chemically defined media with appropriate amounts of manganese and copper. Another method of control is identifying two or more batches of chemically defined media that can be mixed to achieve the specified concentrations when one or more of the batches are outside the specification. Ustekinumab DS or DP produced using the present manufacturing control strategy comprises anti-IL-12/IL-23p40 antibodies, wherein the peak 3 area % of the cIEF electropherogram of the anti-IL-12/IL-23p40 antibodies is ≥39.8% to ≤64.4% and the oligosaccharide profile of the anti-IL-12/IL-23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%. The manufacturing control strategy also ensures that viable cell density (VCD), % viability, and productivity in Stage 2 bioreactors are maintained or improved compared to historical values. In a preferred method, manganese and copper concentrations are determined using ICP-MS and the oligosaccharide profiles are determined using an IPLC method.

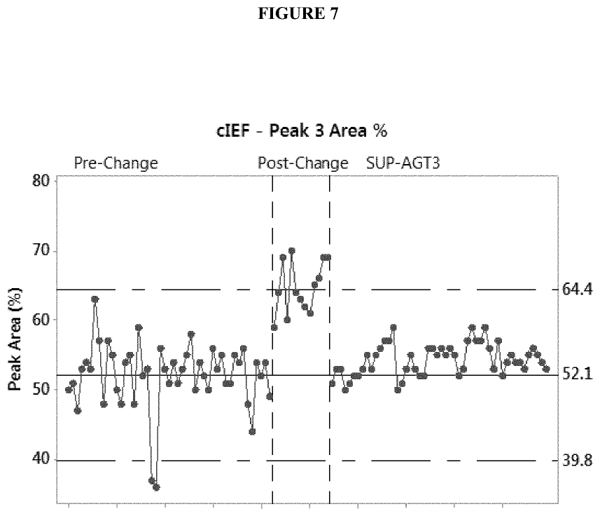

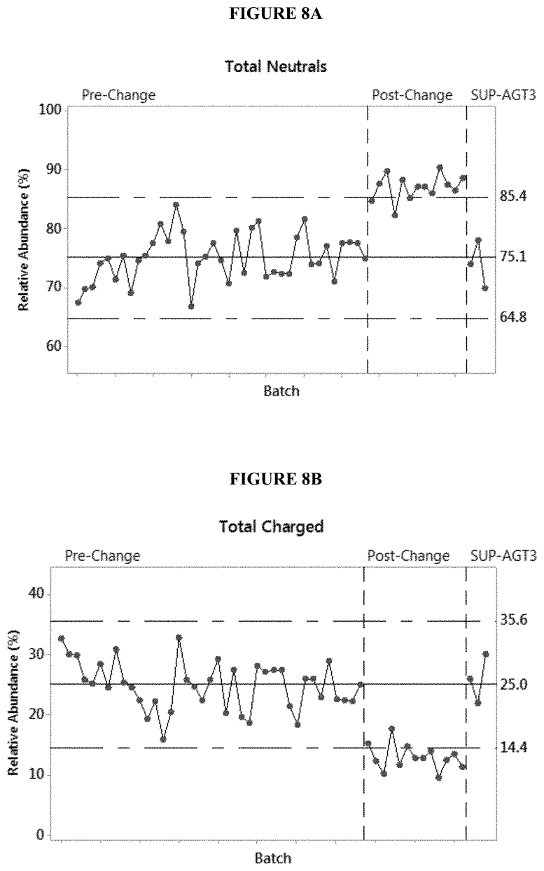

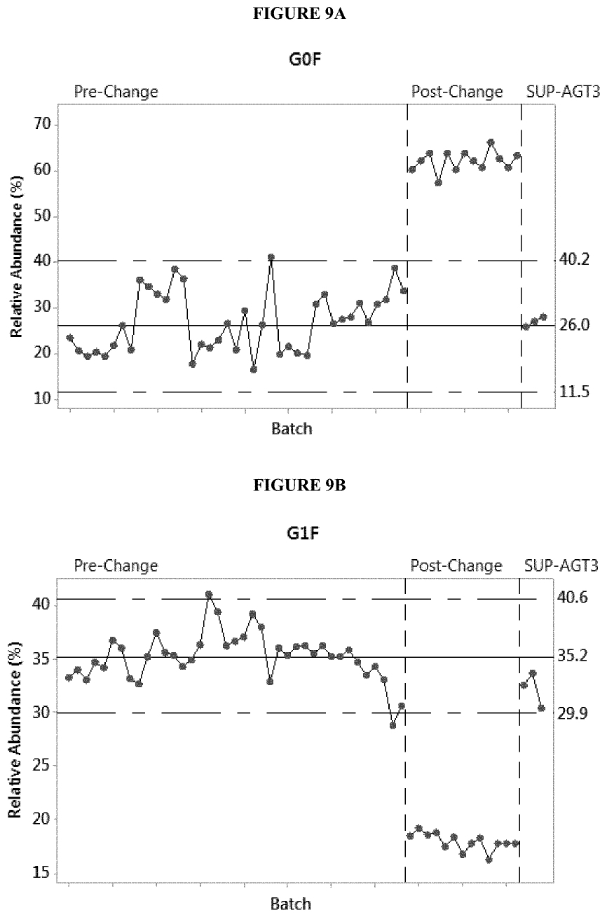

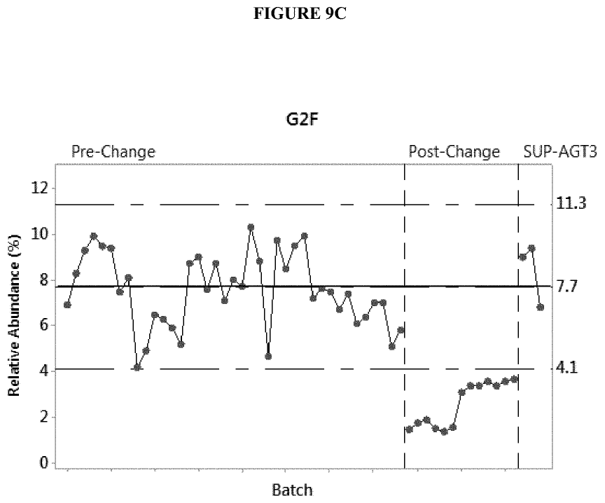

The control strategy was initiated after recognizing atypical trends in production bioreactor performance. Affected production bioreactor batches exhibited shouldering at lower VCD, followed by a dip in the VCD profile compared to historical trends. This change in VCD also affected productivity in terms of the amount of IgG produced. In addition, it was recognized that the cIEF profiles for impacted batches were shifted compared to historical trends. In affected batches, the cIEF shifted towards increased peak 3 area % (an increase in species containing no sialylated glycans). Upon further investigation is was found that and the levels of total neutrals and total charged oligosaccharides were respectively higher and lower than the historical averages (see, e.g., FIG. 7 and FIGS. 8A and 8B). In addition, further evaluation of the individual oligosaccharide species demonstrated that the majority of impacted batches had changes in terminal galactose content (increased agalacto (G0F) and decreased mono-galacto (G1F) and di-galacto (G2F) oligosaccharide species (see, e.g., FIGS. 9A-C, respectively) with a concomitant trend toward decreased sialic acid content in the N-linked oligosaccharide composition resulting in a decrease in negatively charged sialylated species.

After a rigorous investigation, it was concluded that a change in the chemically defined media was the root cause for the change in cIEF peak 3 area %, oligosaccharide profile and shifts in VCD and productivity. More specifically, the investigation demonstrated that surprisingly it was a change in just one of the cell culture media components, FeCl₃.6H₂O (ferric chloride), that was the definitive root cause for the shift in the oligosaccharide profile and shifts in VCD and productivity. In particular, it was determined that a lower trace metal concentration of Mn²⁺ (manganese) in the ferric chloride was the primary root cause for the change in the cIEF peak 3 area % and oligosaccharide profile, and a lower trace metal concentration of Cu²⁺ (copper) in the ferric chloride was the primary root cause for the shifts in VCD and productivity. It was also determined that copper plays a role in determining the oligosaccharide profile and that both manganese and copper concentrations had to be controlled to ensure that the oligosaccharide profile was within specification.

The change in ferric chloride was introduced because of a change in the manufacturing process by the vendor that supplied the ferric chloride that was intended to produce a higher purity iron salt. The investigation revealed that the change resulted in lower trace levels of manganese, chromium and copper that are present as unmeasured impurities in the ferric chloride. Supplementation of media with Mn²⁺ (manganese) and Cr³⁺ (chromium) partially restored the VCD profile, but Cu²⁺ (copper) supplementation was required to fully restore VCD and productivity. Initially, it was suspected that the level of Cr³⁺ (chromium) could be a major factor, but it was determined later with subsequent small-scale studies that the change in the concentration of manganese was the primary contributing factor for the shift in oligosaccharide profile with copper concentration also playing a role and that copper was the primary contributing factor for the changes in VCD and the related change in total productivity.

The manufacturing control strategy remediated the issues associated with changes in the cIEF profile, oligosaccharide profile, VCD and productivity by supplementing the chemically defined media with Mn²⁺ (manganese) and Cu²⁺ (copper). The manufacturing control strategy was implemented in 2 stages at commercial scale. First, the chemically defined media was supplemented with just manganese and chromium to restore the historical levels of these trace metals. This media was referred to as SUP-AGT. In a subsequent change the chemically defined media was supplemented with manganese, chromium and copper to restore all three trace metals to their respective historical levels based on commercial scale results and numerous small-scale studies. This media was referred to as SUP-AGT3.

### Methods

Methods for determining Viable Cell Density (VCD) and % Viability

Total cells per/ml, viable cells/ml (VCD), and % viability are typically determined with a Beckman Coulter Vi-CELL-XR cell viability analyzer using manufacturer provided protocols, software and reagents. Alternatively, a CEDEX automated cell counting system has also been used. It should also be noted, however, that other methods for determining VCD and % viability are well known by those skilled in the art, e.g., using a hemocytometer and trypan blue exclusion.

### Methods for Determining Oligosaccharide Composition

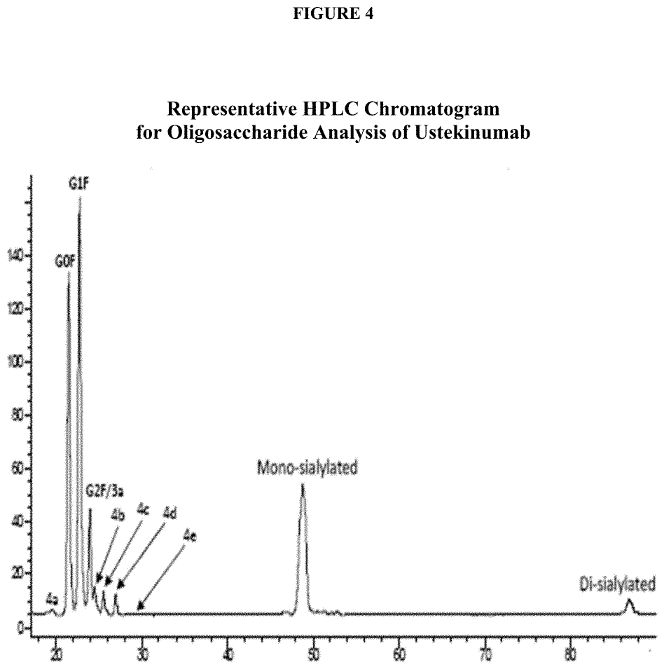

The oligosaccharide composition of ustekinumab is determined with an HPLC method using an Agilent 1100/1200 Series HPLC System with Chemstation/Chemstore software. To quantitate the relative amounts of glycans, the N-linked oligosaccharides are first cleaved from the reduced and denatured test article with N-glycanase (PNGase F). The released glycans are labeled using anthranilic acid, purified by filtration using 0.45-μm nylon filters, and analyzed by HPLC with fluorescence detection. The HPLC chromatogram serves as a map that can be used to identify and quantitate the relative amounts of N-linked oligosaccharides present in the sample. Glycans are identified by co-elution with oligosaccharide standards and by retention time in accordance with historical results from extensive characterizations. A representative HPLC chromatogram for ustekinumab is shown in FIG. 4.

The amount of each glycan is quantitated by peak area integration and expressed as a percentage of total glycan peak area (peak area %). Results are reported for G0F, G1F, G2F, total neutrals, and total charged glycans. Other neutrals are the sum of all integrated peaks between 17 and 35 minutes, excluding the peaks corresponding to G0F, G1F and G2F. Total neutral glycans is the sum of G0F, G1F, G2F and the other neutrals. Total charged glycans is the sum of all mono-sialylated glycan peaks eluting between 42 and 55 minutes and all di-sialylated glycan peaks eluting between 78 and 90 minutes.

A mixture of oligosaccharide standards (G0F, G2F, G2F+N-acetylneuraminic acid (NANA) and G2F+2NANA) is analyzed in parallel as a positive control for the labeling reaction, as standards for peak identification, and as a measure of system suitability. Reconstituted oligosaccharides from Prozyme, G0F (Cat. No. GKC-004301), G2F (Cat. No. GKC-024301), SA1F (Cat. No. GKC-124301), and SA2F (Cat. No. GKC-224301), or equivalent, are used as reference standards. A method blank negative control and pre-labeled G0F standard are also run for system suitability purposes. The following system suitability and assay (test article) acceptance criteria are applied during the performance of the oligosaccharide mapping procedure in order to yield a valid result:

### System Suitability Criteria:

Resolution (USP) between the G0F and G2F peaks in the oligosaccharide standard must be ≥3.0.
Theoretical plate count (tangent method) of the G0F peak in the oligosaccharide standards must be ≥5000.
The total glycan peak area for the ustekinumab reference standard must be ≥1.5 times of the major glycan peak area of the pre-labeled G0F.
If any reference standard glycan peak is off-scale, the reference standard is re-injected with less injection volume
The retention time of G0F peak in the ustekinumab reference standard must be within 0.4 min of the G0F retention time in the oligosaccharide standards.

### Assay Acceptance Criteria:

The method blank must have no detectable peaks that co-elute with assigned oligosaccharide peaks in ustekinumab.
The total glycan peak area of each test article must be ≥1.5 times the major glycan peak area of the pre-labeled G0F standard.
If any sample glycan peak is off-scale, that sample is re-injected with less injection volume, together with pre-labeled G0F, the oligosaccharide standards, Method Blank and reference standard with normal volume.
The retention time for the G0F peak in each test article must be within 0.4 min of the retention time for the G0F peak in the oligosaccharide standards.
If the assay fails to meet any acceptance criteria, the assay is invalidated

### Inductively Coupled Plasma Mass Spectrometry (ICP-MS)

Inductively Coupled Plasma Mass Spectrometry (ICP-MS) is used to quantitate at parts per billion (ppb, μg/liter) trace metal concentrations in the chemically defined media used to produce different batches of ustekinumab. In brief, the method consists of an acid digestion procedure to digest carbon rich sources to carbon dioxide and water before the sample is injected into an ICP-MS instrument such as the NEXION® (mass spectrometer) 350X ICP-MS (PerkinElmer). The wet chemical digestions utilize different acids and oxidizing agents. Preferred combinations include nitric acid (HNO₃), hydrogen peroxide (H₂O₂), and hydrochloric acid (HCl). Analytical methods other than ICP-MS can also be used, e.g., flame atomic absorption spectrometry (FLAA), inductively coupled plasma atomic emission spectrometry (ICP-AES). General information about analytical procedures, sample preparations, and instrumental methods can be found, for example, in EPA method 3050B, “Acid Digestion of Sediments, Sludges, and Soils”, EPA December 1996; EPA memorandum, “use of Hydrochloric Acid (HCL) in digests for ICP-MS analysis”, EPA office for Solid Waste and Emergency Response, Jul. 26, 2003; and United States Pharmacopeia (USP) chapter <233>, Elemental Impurities-Procedures.

Shown below is a customized digestion method that was developed for use in determining metal concentrations in chemically defined media that is analysed by ICP-MS. The method can be adapted to dry media powder or hydrated media samples (Ig sample=1 mL hydrated sample).

### Digestion Method

˜1 g dry samples (±0.5 g, weight recorded to 0.001 g) or ˜1 mL solution samples (±0.5 mL, weight recorded to 0.001 g) are added to digestion vessels (applicable spike solutions are also added at this time)
5.0 mL of 50% v/v HNO₃ (nitric acid) and 2.5 mL concentrated H₂O₂ are added to samples and digestion vessels are then capped immediately with polypropylene watch glasses—H₂O₂ is added slowly to avoid sample bubbling over
samples are heated for 30 minutes at 95° C. (±5° C.)
samples are removed from heat and allowed to cool
2.5 mL concentrated HNO₃ is added and samples are heated for 30 minutes at 95° C. (±5° C.). If brown fumes are generated, indicating oxidation of the sample by HNO₃, the step is repeated over and over until no brown fumes are given off by the sample. No brown fumes is an indication of complete oxidation by HNO₃.
samples are removed from heat and allowed to cool
2.5 mL concentrated HNO₃ and 5 mL concentrated HCl are added and samples are heated for 2 hours at 95° C. (±5° C.)
samples are removed from heat and allowed to cool
total volume of samples is brought to 50 mL with deionized water (DIW) and then samples are ready for analysis
Notes:
   all heating at 95° C. (5° C.) done in reflux, without boiling, with samples capped with polypropylene watch glasses, in pre-heated hot block, e.g., Hotblock® (heating block)
   digestion vials are soaked in 5%/5% v/v HNO₃/HCL overnight and triple rinsed with DIW prior to use
   polypropylene watch glasses are soaked in 5%/5% v/v HNO₃/HCL overnight and triple rinsed with DIW prior to use
plastic tips for pipetting are triple rinsed with reagent prior to use
samples analyzed by ICP-MS within 2 weeks of digestion
methods can also be adapted to automated processes, for example using the Vulcan Automated Digestion and Work-Up System (Questron Technologies Corp.)

### Reagents and Standards

deionized water (DIW) tested to be free of metals, ≥18.0 MΩ
trace metals spike standards from NIST traceable sources
concentrated HNO₃, reagent grade or higher, tested for metals
50% HNO₃ solution—500 mL DIW and slowly added 500 mL HNO₃, solution can be kept for 6 months
concentrated HCL, reagent grade or higher, tested for metals
concentrated (30% v/v) H₂O₂
all DIW, HNO₃, and HCL are tested regularly to ensure there is no contamination

### Capillary Isoelectric Focusing

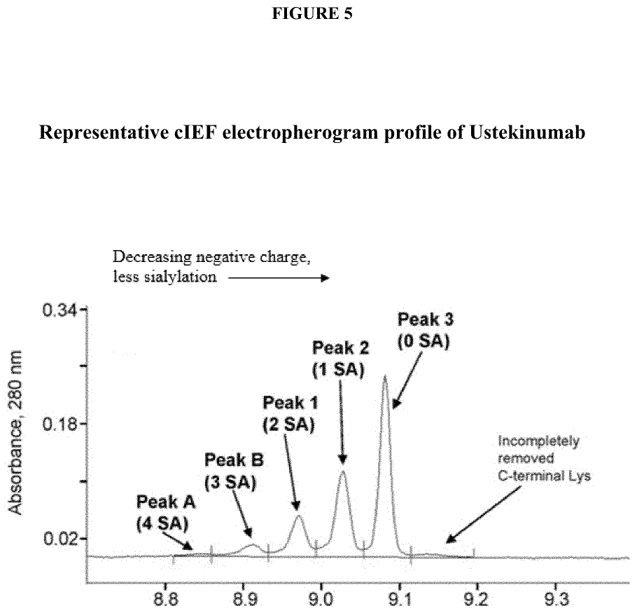

Capillary isoelectric focusing (cIEF) separates proteins on the basis of overall charge or isoelectric point (pI). The method is used to monitor the distribution of charge-based isoforms in ustekinumab. Unlike the gel-based IEF procedures, cIEF provides a quantitative measure of the charged species present. In addition, cIEF shows increased resolution, sensitivity, and reproducibility compared to the gel-based method. The assay is performed on a commercially available imaging cIEF analyzer equipped with an autosampler able to maintain sample temperature ≤10.5° C. in an ambient environment of ≤30° C., such as the Alcott autosampler (GP Instruments, Inc.). The analysis employs an inner wall-coated silica capillary without an outer wall polyimide coating to allow for whole column detection. In addition, an anolyte solution of dilute phosphoric acid and methylcellulose, a catholyte solution of sodium hydroxide and methylcellulose, and a defined mixture of broad range (pH 3-10) and narrow range (pH 8-10.5) ampholytes are used. The assay employs a pre-treatment of both test articles and Reference Standard (RS) with carboxypeptidase B (CPB) which removes the heavy chain C-terminal lysine and eliminates ambiguities introduced by the presence of multiple C-terminal variants. A representative cIEF electropherogram of ustekinumab is shown in FIG. 5.

Before each analysis, the autosampler temperature set-point is set to 4° C. and the autosampler is pre-cooled for at least 30 minutes and the ambient room temperature of the lab is maintained ≤30° C. The pre-treated test article and RS, sample vials, vial inserts, the reagents used in the assay including purified water, the parent solution containing N,N,N′,N′-Tetramethylethylenediamine (TEMED) (which optimizes focusing within the capillary), ampholytes, pI 7.6 and 9.5 markers for internal standards and methylcellulose (MC) are kept on ice for at least 30 minutes before starting sample preparation. The samples are prepared on ice and the time of addition of the parent solution is recorded and exposure to TEMED is controlled. The assay must be completed within 180 minutes after this addition. System suitability controls are injected once and test articles and RS are injected twice following the sequence table below (Table 1):

TABLE 1
Sample Running Sequence

| Sample Name | Sample Vial Position | Number of Injections |
|---|---|---|
| System Suitability | 1 | 1 |
| Blank | 2 | 1 |
| CPB Control | 3 | 1 |
| CPB Treated RS | 4 | 2 |
| CPB Treated Sample 1 | 5 | 2 |
| CPB Treated RS | 6 | 2 |

After the samples are injected into the capillary by a syringe pump, an electric field (3 kV) is applied across the capillary for 8 min, forming a pH gradient, and charge-based isoforms of ustekinumab are separated according to their isoelectric point (pI). The protein isoforms in the capillary are detected by imaging the entire capillary at 280 nm, and the data are presented in the form of an electropherogram as a function of pI value vs A280. Values for pI are assigned by comparison to the internal pI standards (pI 7.6 and 9.5) using the instrument software, and peak areas are determined from the electropherogram using standard data acquisition software. The average pI and average peak area percentage from duplicate injections of all peaks ≥LOQ, the ΔpI value compared to Reference Standard, and percent area peaks are reported.

### Deviating Oligosaccharide Profile

Ustekinumab is N-glycosylated at a single site on each heavy chain, on asparagine 299. These N-linked oligosaccharide structures can be any in a group of biantennary oligosaccharide structures linked to the protein through the primary amine of the asparagine residue, but on ustekinumab they consist primarily of biantennal core-fucosylated species, with galactose and sialic acid heterogeneity. Individual oligosaccharide species include “G0F”, an asialo, agalacto core-fucosylated biantennary glycan, “G1F”, an asialo, mono-galacto core-fucosylated biantennary glycan, and “G2F”, an asialo, di-galacto core-fucosylated biantennary glycan.

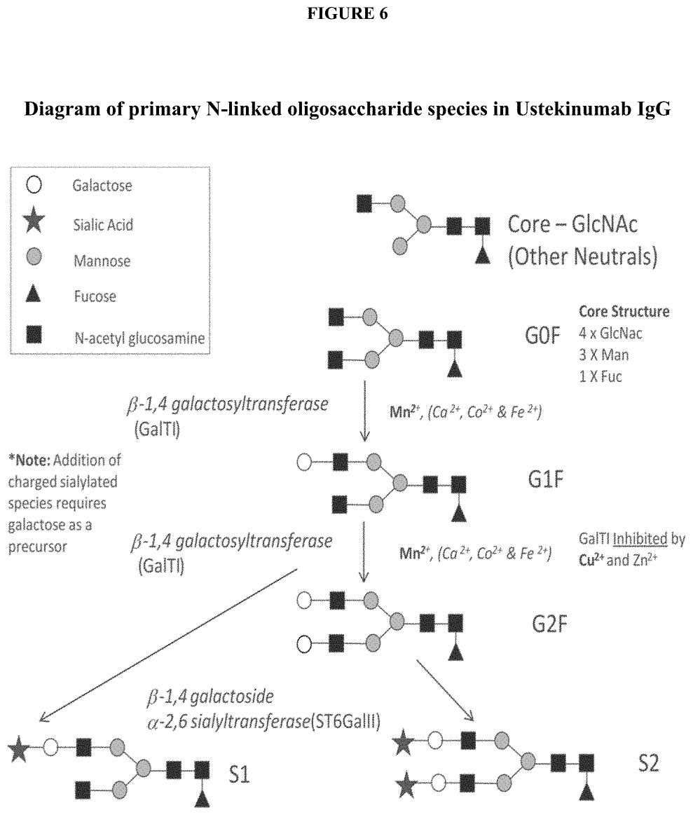

HPLC is an analytical procedure that is deployed to analyze glycosylation of ustekinumab during the manufacturing method. For analysis by HPLC, the glycans are first enzymatically cleaved from the heavy chain and then labeled with a fluorescent label to allow detection. In the method, uncharged peaks for G0F, G1F and G2F can be distinguished, as well as a subset of smaller neutral peaks. Furthermore, peaks for differentially sialylated material can also be observed (FIG. 4). For ustekinumab, there is also a direct relationship between the degree of sialylation on the oligosaccharide structures and the charge heterogeneity as determined by capillary isoelectric focusing (FIG. 5). A diagrammatic overview of some of the primary N-linked oligosaccharide species in ustekinumab IgG is shown in FIG. 6. The role of some of the enzymes in the glycosylation maturation process, including roles of some divalent cations (e.g. Mn²⁺ and Cu²⁺) in these enzymatic processes are also shown.

During the investigation of the impact of the change in culture performance, changes in total neutral and charged oligosaccharides and the levels of individual oligosaccharides of the ustekinumab molecule were evaluated by HPLC. The raw chromatograms and further data analysis indicated that the majority of batches were clearly above and below specifications for total neutral and total charged oligosaccharides, respectively (FIGS. 8A and 8B). Furthermore, there was a shift in the levels of the individual neutral oligosaccharides that were outside the specifications. In fact, all post-change batches of FB showed a shift outside the specifications for the G0F, G1F and G2F species (FIGS. 9A-C, respectively).

Reducing changes in the oligosaccharide profile is critical, because changes in the oligosaccharide profile of a recombinant monoclonal antibody can significantly affect antibody biological functions. For example, biological studies have shown that the distribution of different glycoforms on the Fc region can significantly impact antibody efficacy, stability, and effector function (J. Biosci. Bioeng. 2014 117(5):639-644; Bio-Process Int. 2011, 9(6):48-53; Nat. Rev. Immunol. 2010, 10(5):345-352). In particular, afucosylation (J. Mol. Biol. 368:767-779) and galactosylation (Biotechnol. Prog. 21:1644-1652) can play a huge role in the antibody-dependent cell-mediated cytotoxicity (ADCC) and complement-dependent cytotoxicity (CDC), two important mechanisms by which antibodies mediate killing target cells through the immune function. In addition, high mannose levels have been shown to adversely affect efficacy by increasing clearance of the antibody (Glycobiology. 2011, 21(7):949-959) and sialic acid content can affect anti-inflammatory activity (Antibodies. 2013 2(3):392-414). As a result of these biological consequences from changes in the oligosaccharide profile, regulatory agencies require control of the antibody glycosylation pattern to ensure adherence to lot release specifications for a consistent, safe and effective product.

### Identifying the Problem

After a rigorous and comprehensive investigation, it was concluded that a change in the chemically defined media was the root cause for the shifts in cIEF peak 3 area %, oligosaccharide profiles, VCD and productivity. This chemically defined media is obtained as a powder that is produced by advanced granulation technology (AGT) and is generally referred to herein as AGT. More specifically, the root cause investigation demonstrated that a change in, FeCl₃.6H₂O (ferric chloride), one of greater than ninety components in the AGT, was the definitive root cause for the shifts in cIEF peak 3 area %, oligosaccharide profiles, VCD and productivity. It was determined that the ferric chloride supplier had changed its manufacturing process with the stated intention to produce a higher purity iron salt. Furthermore, the investigation revealed that the change resulted in lower trace levels of manganese, chromium and copper that are present as unmeasured impurities in the ferric chloride. The manganese, chromium and copper are added to the AGT formulation as measured components, but the amounts adventitiously associated with the ferric chloride were not accounted for. Thus, these circumstances resulted in a reduction in the total manganese, chromium, and copper levels in the AGT.

Remediation with Supplemented AGT Media

### Development of SUP-AGT

Preliminary small-scale studies indicated that supplementing the media with manganese and chromium could shift the cIEF peak 3 area % and oligosaccharide profiles back to historical norms (data not shown). Based on those preliminary small-scale studies, a change was implemented to restore historical levels of manganese and chromium to produce commercial batches of ustekinumab by supplementing the AGT with manganese and chromium. The manganese and chromium salts used to supplement the media were already part of the existing media formulation but added at lower concentrations, so it was a matter of supplementing the media with more manganese and chromium salts to produce chemically defined media containing specified limits of Mn²⁺ (manganese) and Cr³⁺ (chromium).

The AGT media supplemented with manganese and chromium is referred to herein as SUP-AGT. The SUP-AGT media powder was produced at large-scale and subsequently used to produce ustekinumab at commercial scale. Evaluation of batches of ustekinumab produced using the SUP-AGT media showed that SUP-AGT did not fully restore VCD and productivity, but it was effective at restoring the historical cIEF Peak 3 area % and oligosaccharide profile, e.g., the levels of G0F, G1F, G2F (data not shown).

The effects on the levels of G0F, G1F, G2F aligns with current literature showing an established relationship with 3-1,4 galactosyltransferase (GalTI) activity, the co-factor Mn²⁺, and glycosylation (FIG. 6, and see e.g., Biotechnol Bioeng. 2007 Feb. 15; 96(3):538-49 and Curr Drug Targets. 2008 April; 9(4):292-309). GalTI is a membrane bound enzyme located in the trans-Golgi membrane. GalTI is activated by Mn²⁺ as a co-factor as it functions within the glycosylation cascade. In this cascade, GalTI adds galactose residues to the core oligosaccharide structure G0F. The reaction products are G1F when one galactose residue is added or G2F when a second galactose residue is added to the N-Acetylglucosamine (GlcNAc) terminals of G0F. The increase in the charged oligosaccharide groups using SUP-AGT aligns with the observed reduction of G0F and the increase of G1F and G2F as the latter two are the main precursors for the formation of SA1 and SA2 oligosaccharides. This sialylation reaction is catalyzed by β-galactoside α-2,6-sialyltransferase (ST6GalII) (FIG. 6) which is a membrane bound enzyme also located in the trans Golgi. Since Mn²⁺ is one of the main co-factors for G1F and G2F formation, and G1F and G2F formation are the main precursors for negatively charged SA1 and SA2 formation, decreased concentrations of Mn²⁺ could have a concomitant effect on the degree of sialylation and charged species. The cIEF peak 3 area % results reflect the changes observed in the charged oligosaccharide groups. The findings from this study support the hypothesis that the changes in the Mn²⁺ concentrations in AGT, due to the changes associated with a purer form of ferric chloride, are the definitive root cause for the changes/trends in cIEF peak 3 area % and oligosaccharide performance.

### Development of SUP-AGT3

The shifts in ustekinumab oligosaccharide profiles were successfully remediated by implementing the change to SUP-AGT media containing higher concentrations of manganese and chromium, however, the change to SUP-AGT did not fully restore VCD and productivity to within historic trends. Analysis during the investigation indicated that reduced Cu⁺ (copper) levels were the most probable root cause for the shifts in VCD, so reduced scale studies were designed to evaluate the effect of supplementing SUP-AGT media with copper.

Trace element solutions containing copper were used to support a series of reduced-scale study experiments with AGT3 spiked with copper added at levels of 0.2 ppb (0.2 μg/liter), 0.5 ppb (0.5 μg/liter), and 0.8 ppb (0.8 μg/liter). The reduced scale studies demonstrated a dose dependent effect, with increasing copper concentrations associated with decreasing levels of GIF and G2F. The results of the reduced scale studies were also compared to historical norms for cell culture performance and it was determined that for each condition tested, the VCD, % viability and productivity were close to the historical means.

The effect of copper concentration on the oligosaccharide profile was also evaluated with small-scale studies, which showed that copper concentration had a significant dose dependent effect on product glycoform profile, with increasing concentrations associated with increasing levels of G0F and decreasing levels of G1F and G2F (data not shown). As noted on FIG. 6, it's known that copper has an inhibitory effect on ß-1-4 galactosyltransferase activity and therefore could have an inhibitory effect on glycosylation (see, e.g., J Biochem Mol Biol. 2002 May 31; 35(3):330-6). It should be noted that this is the opposite of the effect of manganese, which was demonstrated to enhance glycosylation with increasing concentration leading to decreasing levels of G0F and total neutrals. Thus, it was concluded that both copper and manganese concentrations would have to be controlled within ranges that ensure the cIEF peak 3 area % and oligosaccharide profile for ustekinumab is maintained, but also with sufficient copper to support optimal cell growth, viability, and productivity.

With additional small-scale studies and multiple regression analysis of the results from experiments varying the concentrations of both manganese and copper, it was determined that variation in G0F, G1F and G2F could be explained by regression models with manganese and copper as the variables. These data were then used to find optimal concentrations for manganese and copper that would ensure that the ustekinumab produced had a cIEF peak 3 area % and an oligosaccharide profile within specification and also ensure that VCD, % viability, and productivity were within historical norms. The optimal trace metal concentrations for the chemically defined media were determined to be Mn²⁺ (manganese) ≥10.0 μg/liter to ≤35.0 μg/liter and Cu²⁺ (copper) ≥1.0 μg/liter to ≤1.8 μg/liter.

As the analysis about supplementing the media was being completed, a new version of the AGT media was developed by the manufacturer. Relative to the previous version of the media, the manufacturing process for the new media was reported to be optimized around the sequence of addition of polyamine and ethanolamine constituents with the desired effect of improving shelf life stability. The new media is referred to herein as AGT version 3 (AGT3) and is considered a next generation media by Thermo Fisher Inc. This new media, however, has the same characteristics with regard to the source of the ferric chloride. As a result, it also has the same problem of having lower concentrations of the trace metals of Mn²⁺ (manganese) and copper (Cu²⁺). Thus, SUP-AGT3 was created by supplementing the new AGT3 media to contain specified and controlled concentrations of Mn²⁺ (manganese) and Cu²⁺ (copper).

A number of different sources of manganese and copper can be used to achieve the specified limits. Sources of manganese appropriate for use in the present invention include, for example, one or more of MnCl₂, MnSO₄, MnF₂ and MnI₂. Sources of copper appropriate for use in the present invention include, for example, one or more of CuSO₄, CuCl₂, and Cu(OAc)₂. These sources of manganese and copper can be anhydrous or hydrated forms, e.g., dihydrate, tetrahydrate, or pentahydrate forms. A preferred source of manganese includes a combination of MnCl₂ (manganese chloride) and MnSO₄ (manganese sulfate). A preferred source of copper is CuSO₄ (copper sulfate). An advantage in using MnCl₂, MnSO₄, and CuSO₄ as supplements in AGT3 is that these components are already part of the AGT3 formulation at lower concentrations, so no new or different components actually need to be added in the SUP-AGT3 formulation. In summary, specified and controlled limits for manganese and copper were established based on historical data, commercial scale production with SUP-AGT, and the numerous small-scale studies. For Mn²⁺ (manganese), the specified and controlled limits are ≥10.0 μg/liter to ≤35.0 μg/liter and for Cu²⁺ (copper), the limits are ≥1.0 μg/liter to ≤1.8 μg/liter.

Evaluation of Manganese, Chromium, and Copper levels

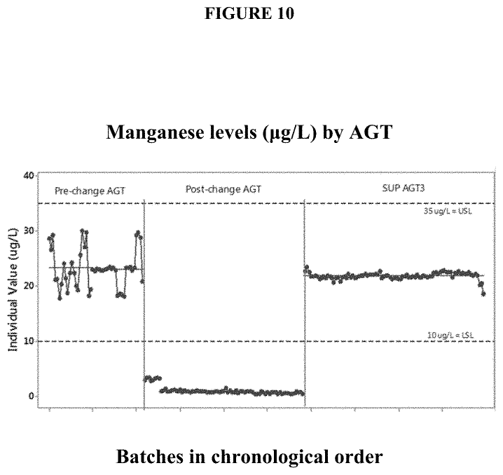

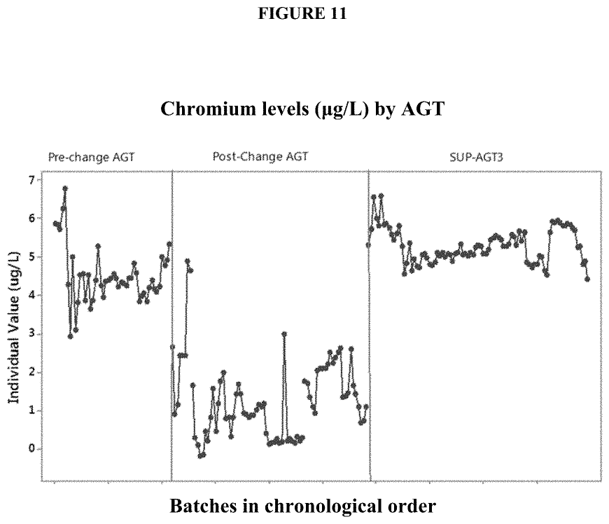

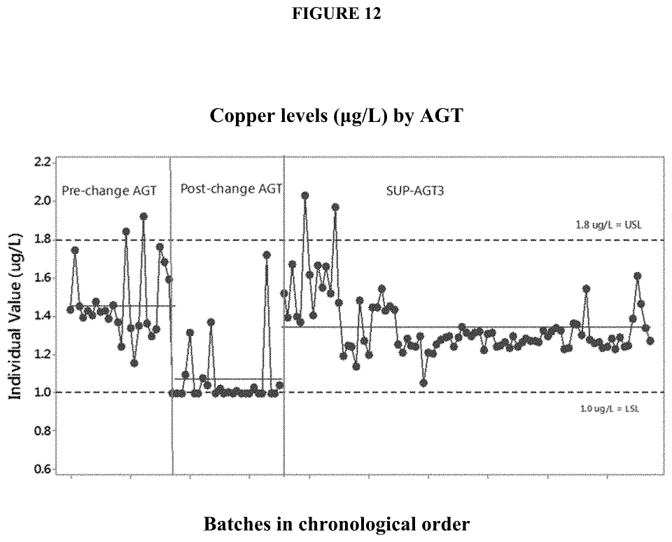

An Inductively Coupled Plasma Mass Spectrometry assay (ICP-MS) was used to evaluate manganese, chromium, and copper levels in different media including, e.g., historical “Pre-change” AGT media before the change in ferric chloride, “Post-change” AGT media used in production of the deviating batches of ustekinumab, and SUP-AGT3. Data for trace metal concentrations for manganese, chromium, and copper levels for different batches of media are shown in FIG. 10, FIG. 11, and FIG. 12, respectively. Note that an individual batch of media is combined with one or more other batches of media for production of ustekinumab at large scale. This has the general effect of homogenizing some of the batch to batch variation of the media, but may not correct for larger variations that are outside of the specification for manganese and copper if the concentrations are not controlled.

The concentration of manganese in all SUP-AGT3 batches was well within the specified and controlled limits of Mn²⁺ (manganese) ≥10.0 μg/liter to ≤35.0 μg/liter that was applied for all SUP-AGT3 batches and also within the range of historical Pre-change AGT batches (FIG. 10). Thus, it was concluded that the concentration of manganese in SUP-AGT3 is well controlled and within the range of historical pre-change AGT.

Except for two early batches of the SUP-AGT3 media, the concentration of copper was also well within the specified and controlled limits of Cu²⁺ (copper) ≥1.0 μg/liter to ≤1.8 μg/liter (FIG. 12). The out of specification situation for the two affected batches of SUP-AGT3 media was easily remedied by mixing those batches with other different batches of SUP-AGT3 media to obtain media for use in manufacturing ustekinumab that was within the specification. Thus, it was concluded that the copper concentration for SUP-AGT3 is well controlled. Note that the two batches of historical pre-change AGT media that were out of specification were combined with other batches of pre-change AGT media as a matter of routine, so the combined batches were also within the specification during production of ustekinumab with historical pre-change AGT. Also note that many of the Post-change AGT batches had values less than the limit of detection of the assay for copper (1 μg/liter). These values are represented graphically as 1 μg/liter in FIG. 12.

Commercial Batch Manufacture with SUP-AGT3

### Oligosaccharide Profile Using SUP-AGT3

Based on the positive results for trace metal analysis, SUP-AGT3 was introduced into the ustekinumab commercial manufacturing process at different production facilities using 500 liter or 1000 liter bioreactors. The medium was used for all cell culture steps, from Stage 1 (preculture and seed bioreactor) through to Stage 2 (production bioreactor process) and product generated for the different bioreactors was used to produce different batches of ustekinumab. As shown in FIG. 7, FIGS. 8A and 8B, and FIGS. 9A-C, ustekinumab produced with SUP-AGT3 comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%. Furthermore, the peak 3 area % of the capillary isoelectric focusing (cIEF) electropherogram of ustekinumab produced with SUP-AGT3 is ≥39.8% to ≤64.4%.

### Cell Viability and Productivity Using SUP-AGT3

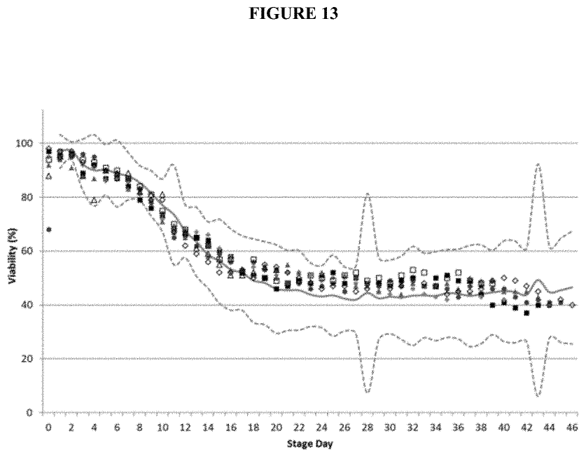

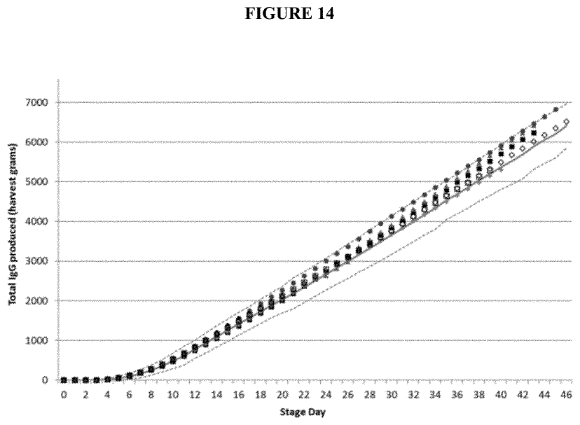

Use of SUP-AGT3 to produce ustekinumab in the commercial manufacturing process also restored or improved cell viability and productivity compared to historical norms. See, e.g., FIG. 13 shows Stage 2 cell culture viability for cells for different SUP-AGT3 batches of ustekinumab and FIG. 14 shows Stage 2 cell culture productivity for different SUP-AGT3 batches of ustekinumab.

### CONCLUSION

Thus, as described supra, a manufacturing control strategy was developed to maintain consistent drug substance (DS) and drug product (DP) characteristics of ustekinumab with regard to oligosaccharide profile and also to control cell viability and productivity during large-scale production. A DS or DP produced by the methods of the present invention comprises anti-IL-12/IL-23p40 antibodies having a heavy chain (HC) comprising SEQ ID NO:10 and a light chain (LC) comprising SEQ ID NO:11; a heavy chain variable domain amino acid sequence of SEQ ID NO:7; and a light chain variable domain amino acid sequence of SEQ ID NO:8; the heavy chain CDR amino acid sequences of SEQ ID NO:1, SEQ ID NO:2, and SEQ ID NO:3; and the light chain CDR amino acid sequences of SEQ ID NO:4, SEQ ID NO:5, and SEQ ID NO:6; wherein the oligosaccharide profile of the anti-IL-12/IL-23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%. Furthermore, the peak 3 area % of the capillary isoelectric focusing (cIEF) electropherogram of the anti-IL-12/IL-23p40 antibodies is ≥39.8% to ≤64.4%. The oligosaccharide profile of DS and DP is controlled by culturing eukaryotic cells in chemically defined media specified and controlled to contain trace metal concentrations consisting of Mn²⁺ (manganese) ≥10.0 μg/liter to 35.0 μg/liter and Cu²⁺ (copper) ≥1.0 μg/liter to ≤1.8 μg/liter; and expressing the anti-IL-12/IL-23p40 antibodies in the eukaryotic cells. Furthermore, the manufacturing control strategy also maintains or improves cell viability and productivity of cells during ustekinumab production as compared to historical norms. Ustekinumab oligosaccharide profiles were determined using an HPLC method and manganese and copper concentrations of the media were measured using inductively coupled plasma mass spectrometry (ICP-MS).

What is claimed:

1. An anti-IL-12/23p40 antibody comprising: (i) heavy chain CDR amino acid sequences of SEQ ID NO:1, SEQ ID NO:2, and SEQ ID NO:3; and (ii) light chain CDR amino acid sequences of SEQ ID NO:4, SEQ ID NO:5, and SEQ ID NO:6, wherein the oligosaccharide profile of the anti-IL-12/IL-23p40 antibody comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, and wherein the anti-IL-12/IL-23p40 antibody is produced by a method of manufacture that controls the oligosaccharide profile of the anti-IL-12/23p40 antibody, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/IL-23p40 antibody in the eukaryotic cells, wherein the concentrations of manganese and copper are effective at controlling the oligosaccharide profile of the anti-IL-12/23p40 antibody.

2. An anti-IL-12/23p40 antibody comprising: (i) a heavy chain variable domain amino acid sequence of SEQ ID NO:7; and (ii) a light chain variable domain amino acid sequence of SEQ ID NO:8, wherein the oligosaccharide profile of the anti-IL-12/IL-23p40 antibody comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, and wherein the anti-IL-12/IL-23p40 antibody is produced by a method of manufacture that controls the oligosaccharide profile of the anti-IL-12/23p40 antibody, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/IL-23p40 antibody in the eukaryotic cells, wherein the concentrations of manganese and copper are effective at controlling the oligosaccharide profile of the anti-IL-12/23p40 antibody.

3. An anti-IL-12/23p40 antibody comprising: (i) a heavy chain amino acid sequence of SEQ ID NO:10; and (ii) a light chain amino acid sequence of SEQ ID NO:11, wherein the oligosaccharide profile of the anti-IL-12/IL-23p40 antibody comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, and wherein the anti-IL-12/IL-23p40 antibody is produced by a method of manufacture that controls the oligosaccharide profile of the anti-IL-12/23p40 antibody, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/IL-23p40 antibody in the eukaryotic cells, wherein the concentrations of manganese and copper are effective at controlling the oligosaccharide profile of the anti-IL-12/23p40 antibody.

4. The anti-IL-12/23p40 antibody of claim 1, wherein the anti-IL-12/23p40 antibody comprises a follow-on biologic.

5. A method of manufacture for producing a drug substance (DS) or drug product (DP) comprising anti-IL-12/IL-23p40 antibodies comprising a heavy chain of the amino acid sequence of SEQ ID NO:10 and a light chain of the amino acid sequence of SEQ ID NO:11, wherein the oligosaccharide profile of the anti-IL-12/IL-23p40 antibodies is controlled and the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F ≥29.9% to ≤40.6%, and G2F ≥4.1% to ≤11.3%, the method comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/IL-23p40 antibodies in the eukaryotic cells, wherein the concentrations of manganese and copper are effective at controlling the oligosaccharide profile of the anti-IL-12/23p40 antibodies.

6. The method of manufacture of claim 5, wherein the peak 3 area % of the capillary isoelectric focusing (cIEF) electropherogram of the anti-IL-12/IL-23p40 antibodies is ≥39.8% to ≤64.4%.

7. The method of manufacture of claim 5, wherein the specified trace metal concentrations of manganese and copper in the chemically defined medium are determined using inductively coupled plasma mass spectrometry (ICP-MS).

8. The method of claim 5, wherein the trace metal concentrations of manganese and copper in the chemically defined medium are controlled by supplementing the chemically defined medium with one or more sources of manganese and copper, wherein the one or more sources of manganese are selected from the group consisting of: MnCl₂, MnSO₄, MnF₂ and MnI₂ and the one or more sources of copper are selected from the group consisting of: CuSO₄, CuCl₂, and Cu(OAc)₂.

9. The method of manufacture of claim 5, wherein the oligosaccharide species are determined by high pressure liquid chromatography (HPLC).

10. The method of manufacture of claim 5, wherein the eukaryotic cells are selected from the group consisting of: Chinese hamster vary cells (CHO cells), human retinal cells (PER.C6 cells), and mouse myeloma cells (NS0 cells and Sp2/0 cells).

11. The method of manufacture of claim 5, wherein the anti-IL-12/23p40 antibodies comprise a follow-on biologic.

12. A composition comprising anti-IL-12/IL-23p40 antibodies comprising a heavy chain of the amino acid sequence of SEQ ID NO:10 and a light chain of the amino acid sequence of SEQ ID NO:11, wherein the oligosaccharide profile of the anti-IL-12/23p40 antibodies comprises total neutral oligosaccharide species ≥64.8% to ≤85.4%, total charged oligosaccharide species ≥14.4% to ≤35.6%, and individual neutral oligosaccharide species G0F ≥11.5% to ≤40.2%, G1F=≥29.9% to ≤40.6%, and G2F ≥4.1% to ≤11.3%, and wherein the anti-IL-12/IL-23p40 antibodies are produced by a method of manufacture that controls the oligosaccharide profile of the anti-IL-12/23p40 antibodies, the method of manufacture comprising: culturing eukaryotic cells in chemically defined medium controlled to contain specified trace metal concentrations of manganese and copper consisting of, Mn²⁺ (manganese) ≥10.0 μg/liter to ≤35.0 μg/liter and Cu²⁺ (copper) ≥1.0 μg/liter to ≤1.8 μg/liter; and expressing the anti-IL-12/IL-23p40 antibodies in the eukaryotic cells, wherein the concentrations of manganese and copper are effective at controlling the oligosaccharide profile of the anti-IL-12/23p40 antibodies.

13. The composition of claim 12, wherein the peak 3 area % of the capillary isoelectric focusing (cIEF) electropherogram of the anti-IL-12/IL-23p40 antibodies is ≥39.8% to ≤64.4%.

14. The composition of claim 12, wherein the specified trace metal concentrations of manganese and copper in the chemically defined medium are determined using inductively coupled plasma mass spectrometry (ICP-MS).

15. The composition of claim 12, wherein the specified trace metal concentrations of manganese and copper in the chemically defined medium are controlled by supplementing the chemically defined medium with one or more sources of manganese and copper, wherein the one or more sources of manganese are selected from the group consisting of: MnCl₂, MnSO₄, MnF₂ and MnI₂ and the one or more sources of copper are selected from the group consisting of: CuSO₄, CuCl₂, and Cu(OAc)₂.

16. The composition of claim 12, wherein the oligosaccharide species are determined by high pressure liquid chromatography (HPLC).

17. The composition of claim 12, wherein the eukaryotic cells are selected from the group consisting of: Chinese hamster vary cells (CHO cells), human retinal cells (PER.C6 cells), and mouse myeloma cells (NS0 cells and Sp2/0 cells).

18. The composition of claim 12, wherein the anti-IL-12/23p40 antibodies comprise a follow-on biologic.


SEQUENCE LISTING (11 sequences)

SEQ ID NO: 1 - protein, 5 aa, Homo sapiens
TYWLG

SEQ ID NO: 2 - protein, 17 aa, Homo sapiens
IMSPVDSDIRYSPSFQG

SEQ ID NO: 3 - protein, 10 aa, Homo sapiens
RRPGQGYFDF

SEQ ID NO: 4 - protein, 11 aa, Homo sapiens
RASQGISSWLA

SEQ ID NO: 5 - protein, 7 aa, Homo sapiens
AASSLQS

SEQ ID NO: 6 - protein, 9 aa, Homo sapiens
QQYNIYPYT

SEQ ID NO: 7 - protein, 119 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 108 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 9 - protein, 503 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 449 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC